(19) United States
(12) Patent Application Publication
(10) Pub. No.: US 2007/0032877 A1
(43) Pub. Date: Feb. 8, 2007

(54) Coated ceramic total joint arthroplasty and method of making same

(76) Inventor: Leo A. Whiteside, Chesterfield, MO (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/499,074

(22) Filed: Aug. 4, 2006

Related U.S. Application Data
(60) Provisional application No. 60/705,855, filed on Aug. 5, 2005.

(51) Int. Cl. A61F 2/32

(52) U.S. Cl. 623/22.15; 623/23.6

(57) ABSTRACT

A total joint arthroplasty is described having a first bearing member (e.g., the femoral head of the femoral component of a total arthroplasty hip joint) and a second bearing member (e.g., an acetabular component) cooperable with the first bearing member with the articulating surfaces of the bearing members in engagement with one another, and wherein at least one of the bearing members has a substrate of a magnesia-stabilized zirconia ceramic material and a bearing surface having a carbon coating applied to the bearing surface.

### CROSS-REFERENCE TO RELATED APPLICATIONS

This application claims priority under 35 U.S.C. § 119(e) of U.S. Provisional Patent Application No. 60/705,855 filed Aug. 5, 2006, which is incorporated herein by reference.

### STATEMENT REGARDING FEDERALLY SPONSORED RESEARCH OR DEVELOPMENT

Not Applicable.

### BACKGROUND OF THE DISCLOSURE

This disclosure relates to a total joint arthroplasty (i.e., the surgical reconstruction or replacement of a malformed or degenerated joint with a prosthetic joint), and, in particular, to a joint prosthesis employing hard ceramic substrates, such as magnesia-stabilized zirconia, with a diamond coating applied to one or more of the bearing surfaces of the substrates.

Over the past several decades, great strides have taken place in joint replacement. However, particularly in knee and hip replacements, problems involving wear persist. The most common cause of failure in modern total hip replacement is wear of the articulating bearing surfaces.

Most bearing surfaces are composed of metal, such as cobalt-chromium alloy stainless steel, against polyethylene. The metal can be highly polished, but it oxides as time passes, and thus roughens and releases highly abrasive oxide particles and carbide particle inclusions into the joint. Improvements in the polyethylene, such as cross-linking and compression molding, have improved the metal-on-polyethylene bearing surfaces, but the time related deterioration of the metal surface continues to limit the effectiveness of these types of implants, especially in young, active patients. Ceramic surfaces are an alternative to metal, and may have the advantage of negligible corrosion and lower wear of the polyethylene counterface. However, the ceramic surface can also deteriorate and roughen as it ages. An improved hard bearing surface that did not corrode nor roughen could be highly advantageous when articulating against a polyethylene, especially in active patients.

Some of these issues have been addressed with zirconium alloy metal that is oxidized for a zirconium oxide ceramic surface. Although these oxidized zirconium surfaces are more resistant to wear and corrosion than metal surfaces, and are stronger than standard ceramic implants, they have the disadvantage of being susceptible to scratching by third body particles.

A total joint implant that could be made very strong and hard with a very smooth surface that is resistant to abrasion and corrosion would be a major improvement over currently available devices. Such a device would produce less wear when articulating against polyethylene and would have the potential of lasting the lifetime of a young and active patient.

One solution to the problem of wear in younger and more active patients has been to use two hard surfaces articulating on each other. Metal-on-metal articulations have advantages of great strength, low wear, and excellent durability. However, these metal-on-metal articulation surfaces release potentially toxic metal ions and occasionally are subjected to high friction and wear. Ceramic-on-ceramic articulating surfaces exhibit low wear and friction characteristics and produce low toxicity particulate debris. Alumina ceramics appear to be the leading candidate materials in the field of ceramic-on-ceramic bearing surfaces because the articular surfaces of alumina ceramic are compatible with one another, and alumina ceramic materials can perform millions of cycles with minimal wear. However, the strength of alumina ceramic is a clinical problem, and has not been completely solved. Moreover, making alumina ceramic femoral heads for total hip replacement appliances in smaller sizes, and using alumina (and other ceramic materials) in thin components for fitting smaller hips is not practical because of the poor tensile strength of this material. Also, alumina ceramic can lose crystals from its surface. These alumina ceramic crystals are very hard and abrasive and can lead to accelerated third-body wear and catastrophic failure. Fractures of alumina ceramic femoral heads have been reported with high frequency.

Zirconia ceramics have the advantage of higher strength and toughness compared to alumina ceramics. Zirconia ceramics can be polished to a very smooth surface, and the strength of zirconia is excellent for use with total hip arthroplasty. However, zirconia ceramics have had a problem with composition leading to late surface roughening, fracture, and catastrophic failure. The commonly used yittria-stablized ceramic material is formulated to assume a tetragonal crystalline structure that has great strength and durability. Under conditions of aging and heating, the tetragonal crystalline structure can degrade to a monoclinic structure that occupies larger space. This creates a roughening of the surface, deformation of the spherical shape of the femoral head, and weakening of the material.

One of the solutions to high wear in total joint arthroplasty is to use an extremely hard, low friction, and bio-inert coating on a metallic surface. Diamond coating is one of the potential candidates for creating such hard and durable surfaces. Diamond coating on metal femoral head and on metal acetabular socket components have performed well in laboratory settings, and are currently under limited investigational human use. Examples of diamond coated metal hip replacement components are disclosed in U.S. Pat. Nos. 5,645,601, 6,610,095 and 6,800,095.

One of the potential disadvantages of diamond coated metallic substrates is damage to the underlying, relatively soft metallic substrate. The diamond coating is very thin, typically about 10 microns or less, and can be deformed by a hard third body that becomes trapped between two diamond bearing surfaces, such as a hard debris particle trapped between a diamond coated femoral head and a diamond coated acetabular socket. This debris particle can deform and tear, crack or puncture the thin diamond coating due to softness of the metal substrate, leading to a scratch or other void in the diamond coating that will release diamond and metal particles, potentially precipitating catastrophic wear. Additionally, debris particles can cause the diamond coating to fail locally thus causing diamond particles to be present between the bearing surfaces of the ball and the acetabular socket. Diamond debris particles are extremely hard and result in extremely high point loading on the diamond surface when the joint is subjected to normal usage loads. This point loading will cause the relatively soft metallic substrate to deform in the local area of the debris particle thus causing further degradation of the diamond coating, and, eventually, causing damage to the joint.

Another disadvantage of diamond coating on a metal substrate arises from differences in stiffness of the diamond and metallic materials. Compression loading of two layers of different elastic modulus results in shear stress at the interface because of different coefficients of deformation of the materials. This dislocation is addressed by interfacial layers to strengthen the interfaces and to prevent deformation.

The carbon vapor deposition method of applying diamond coatings maybe used to produce an amorphous diamond-like carbon coating on a variety of surfaces. One of the characteristics of this process is the presence of “pin holes” that measure about 3-10 microns in diameter. A potential disadvantage of applying such a diamond-like coating to a metal substrate is that such “pinholes” may permit corrosive joint fluid to penetrate through the pinholes in the inert diamond coating to the metal substrate. This may result in corrosion of the metal substrate that can undermine the diamond-like coating and lead to flaking (delamination) of the coating from the metal substrate and finally to catastrophic wear and failure.

An ideal joint replacement implant would have a very hard and strong substrate material with stable structure, and a highly inert surface that produces extremely low wear and low friction, but does not have the disadvantage of potential scratching due to deformation of the substrate material, nor the potential of failure due to undermining and delamination of the diamond (or diamond-like) coating caused by substrate corrosion.

### BRIEF SUMMARY OF THE DISCLOSURE

Among the several objects and advantages of a total arthroplasty component disclosed herein are: the provision of a diamond (or diamond-like) coating applied to a ceramic substrate where the substrate is very hard (compared to metallic substrates), is very tough, is highly inert in biological fluids, and has a stable crystalline structure that does not degrade over time or when exposed in a biological environment;

The provision of such a total arthroplasty component having low wear and friction properties;

The provision of such a total arthroplasty component having a substrate impervious to corrosion from joint fluid thus minimizing the tendency of the diamond coating to delaminate from the substrate;

The provision of such a total arthroplasty component having a ceramic substrate that is stable to aging and heating thus providing an excellent substrate for being diamond coated;

The provision of such a total arthroplasty component utilizing magnesia stabilized zirconia as the substrate;

The provision of such a total arthroplasty component that may be diamond coated using known coating technologies and techniques;

The provision of such a total arthroplasty component that is resistant to corrosion and failure, even if such diamond coating techniques leave pinholes and voids in the diamond coating;

The provision of such a total arthroplasty component which can be made relatively thin so as to form a thin shell that may be fitted into a shell that in turn may be press fit into a socket;

The provision of such a total arthroplasty component that is tough, that will not fracture in normal usage, and that resists chipping;

The provision of such a total arthroplasty joint in which two joint components that articulate on one another are made of magnesia-stabilized zirconia, wherein one or both of the bearing surfaces of these components have a diamond coating applied thereto thus protecting the magnesia-stabilized bearing components from directly contacting one another such that the diamond coating provides highly wear-resistant bearing surfaces that have a very low coefficient of friction;

The provision of a method of making such total arthroplasty components utilizing magnesia-stabilized zirconia substrates and a diamond coating on the bearing surface of the substrates where the diamond coating is readily applied using known techniques; and

The provision of such a total arthroplasty components that are durable, readily fabricated, economical to fabricate, relatively easy to install, and have a long service life.

It will be appreciated that not all of the above objects and advantages need be exhibited in each of the claims of this disclosure. Other objects and features of this disclosure will be in part apparent and in part pointed out hereinafter.

### BRIEF DESCRIPTION OF THE SEVERAL VIEWS OF THE DRAWINGS

FIG. 1 is a side elevational part-cross-sectional view of a prior art prosthetic hip joint in which a diamond coating is applied to the outer surface of a metal substrate femoral ball of a femoral component and to the inner spherical surface of a metal acetabular component;

FIG. 2 is a side elevational part-cross-sectional view of prosthetic joint appliance (e.g., a hip joint appliance) of the present disclosure comprising a first (femoral) bearing member of magnesia stabilized zirconia to which a thin carbon coating (shown in exaggerated scale) is applied, and a second (an acetabular cup) bearing member adapted to be secured to a patient by bone screws where the second bearing member (cup) receives a part-spherical liner of magnesia-stabilized zirconia with the inner surface of the liner having a thin carbon coating applied thereto so that with the first bearing member received therein the carbon coating on the first bearing member and the carbon coating on the second bearing member constitute bearing surfaces in engagement with one another;

FIGS. 3A-3H illustrate a total arthroplasty appliance (e.g., a knee joint appliance) having carbon coatings applied to at least the bearing surfaces of the femoral (FIGS. 3A-3D) and tibial (FIGS. 3E-3H) components of the knee joint appliance wherein the femoral and tibial components have a magnesia stabilized zirconia substrate;

4 illustrates light microscopy images of the typical surface of: A. uncoated CoCr; B. coated CoCr; C. uncoated Mg-PSZ; and D. coated Mg-PSZ femoral heads. Magnification=100×; bars represent 50 μm;

FIG. 5 illustrates a surface plot illustrating topography of a non-coated CoCr femoral head (Sa=18 nm), with localized depressions (diameter=20-30 μm; depth=60-100 nm) present;

FIG. 6 illustrates a surface plot illustrating topography of a diamond-like coated CoCr femoral head (Sa=12 nm), with localized depressions (diameter=10-15 μm; depth=100-125 nm) present;

FIG. 7 illustrates a surface plot illustrating topography of a non-coated Mg-PSZ femoral head (Sa=5.7 nm), wherein the deep “scratch” has a depth of about 20 nm, is about 4 μm wide, and about 20 μm long;

FIG. 8 illustrates a surface plot illustrating topography of a diamond-like coated Mg-PSZ femoral head (Sa=5.3 nm), wherein the most significant depression above has a conical profile but is very shallow, with a diameter of about 30 μm and a depth of about 25 nm;

FIG. 9 illustrates a surface plot illustrating topography of the articulating surface of an Oxinium TKA femoral component (Sa=19.3 nm). The surface was characterized by long scratches such as the one shown; scratches were typically about 10 μm wide and about 150 nm deep. Note that the vertical scale is 600.6 instead of 360 nm, although the vertical axis was not amplified to show detail;

FIG. 10 illustrates successful Vickers indentations into: A. uncoated CoCr and B. uncoated Mg-PSZ femoral heads, both made using a load of 4.91 N. The slight asymmetry of the indents is due to the curvature of the surface, but all indents were verified to conform to ASTM C1327.

FIG. 11 illustrates successful Vickers indentations into: A. coated CoCr and B. coated Mg-PSZ femoral heads, both made using a load of 2.94 N. The relatively softer CoCr substrate deformed fast enough during loading to allow the coating to crack and fail cohesively, as evidenced by the successive circumferential cracks. In contrast, the coating on the Mg-PSZ head remained intact as its harder substrate deformed, with cracks rarely present. Magnification=40×; bars represent 25 μm;

FIG. 12 illustrates a surface plot of a non-coated CoCr femoral head;

FIG. 13 illustrates a surface plot of a diamond-like coated CoCr femoral head. The round positive features (each less than 50 nm high and less than 1 μm wide) were found on additional AFM scans, even after gently cleaning with alcohol, and may be caused by dust;

FIG. 14 illustrates a surface plot of a non-coated Mg-PSZ femoral head;

FIG. 15 illustrates a surface plot of a diamond-like coated Mg-PSZ femoral head;

FIG. 16 illustrates graphical comparison of the scratch tracks of uncoated CoCr (left side) and diamond-like coated CoCr (right side), at loads of 0.50 N and 0.75 N (top to bottom). Each of the four images are about the same size (469 μm×188 μm), and were obtained by light microscopy at the same scale (200×; the bar in the corner represents 50 μm);

FIG. 17 illustrates a detail of scratch tracks in uncoated (left) and diamond-like coated CoCr (right) femoral heads (0.75 N load, 500×). Abundant pile-up is visible around edge of the scratch on the uncoated CoCr specimen, while small cracks (evidence of cohesive fracture) are visible in the diamond-like coated CoCr specimen's surface. This suggests that the substrate is yielding faster than the coating;

FIG. 18 illustrates a graphical comparison of the scratch tracks of uncoated Mg-PSZ (left side) and diamond-like coated Mg-PSZ (right side), at loads of 0.50 N, 0.75 N, 1.00 N, and 1.25 N (top to bottom). Each of the eight images are about the same size (469 μm×188 μm), and were obtained by light microscopy at the same scale (200×; the bar in the corner represents 50 μm);

FIG. 19 illustrates a diamond-like coated CoCr femoral head (light microscopy, 200×). At a load of 1 N, the coating delaminated from the substrate. The “base” of the wear track is about 19 μm wide;

FIG. 20 illustrates a diamond-like coated Mg-PSZ head at fracture (1.5 N load; light microscopy, 200×). The “base” wear track is about 11 μm wide, and appears to zig-zag across the surface, suggesting that the coating was strong enough to deflect the stylus laterally as it fractured;

FIG. 21 illustrates a constant-load scratch tests on Oxinium TKA femoral component: left, 1 N at 200× (wear track is about 17 μm wide); right, 3.25 N at 200×(wear track is about 55 μm wide);

FIG. 22 illustrates a graphical comparison of the scratch tracks of Oxinium (left side) and Mg-PSZ (right side), at loads of 0.50 N, 0.75 N, 1.00 N, and 1.25 N (top to bottom). Each of the eight images are about the same size (469 μm×188 μm), and were obtained by light microscopy at the same scale (200×; the bar in the corner represents 50 μm). At the loads shown, diamond-like coated Mg-PSZ is clearly more resistant to abrasive wear than Oxinium;

FIG. 23 illustrates a typical cohesive fracture (left, wedging spallation at 10.6 N) and adhesive fracture (right, delamination at 38.3N) of a diamond-like coated CoCr femoral head (light microscopy, 200×). Scratch direction was from left to right;

FIG. 24 illustrates a typical cohesive fracture (left, forward tensile cracks with a small chip at 40.8 N) and adhesive fracture (right, recovery spallation at 44.6 N) of diamond-like coated Mg-PSZ femoral heads (light microscopy, 200×). Scratch direction was from left to right;

FIG. 25 illustrates a typical cohesive fracture (left, chipping at 26.8 N) and adhesive fracture (right, delamination at 38.5 N) of an Oxinium femoral component (light microscopy, 200×). Scratch direction was from left to right;

FIG. 26 illustrates a typical diamond-like coated Mg-PSZ femoral heads at 80 N (light microscopy, 200×). A small amount of chipping occurred along the edges of the scratch track, but the lack of particulate debris associated with other diamond-like coating fractures (see previous constant-load scratch test figures) suggests that the coating did not delaminate, but rather was pushed into the substrate (recovery spallation). Scratch direction was from left to right;

FIG. 27 illustrates a graph showing a scratch width and scratch depth profile, along with pile-up and chipping along the scratch track, wherein the profile was extracted from white-light interferometry scans at 16×, at a load of 30 N (lateral to the scratch direction);

FIG. 28 illustrates a graph showing a scratch width and scratch depth profile, along with pile-up and chipping along the scratch track, wherein the profile was extracted from white-light interferometry scans at 16×, at a load of 30N (lateral to the scratch direction, detail);

FIG. 29 illustrates a graph showing a scratch width and scratch depth profile, along with pile-up and chipping along the scratch track, wherein the profile was extracted from white-light interferometry scans at 16×, at a load of (parallel to the scratch direction);

FIG. 30 illustrates a graph showing a scratch width and scratch depth profile, along with pile-up and chipping along the scratch track, wherein the profile was extracted from white-light interferometry scans at 16×, at a load of 60 N (parallel to the scratch direction);

FIG. 31 illustrates an optical profilometry image of a CoCr (no coat) specimen, 60 N;

FIG. 32 illustrates an optical profilometry image of a Mg-PSZ (no coat) specimen, 60 N;

FIG. 33 illustrates an optical profilometry image of a diamond-like coated CoCr, specimen 60 N;

FIG. 34 illustrates an optical profilometry image of a diamond-like coated Mg-PSZ specimen, 60 N; and

FIG. 35 illustrates an optical profilometry image of an Oxinium specimen, 60 N.

Corresponding reference numerals will be used throughout the several figures of the drawings.

### DETAILED DESCRIPTION OF A PREFERRED EMBODIMENT

The following detailed description illustrates the disclosure by way of example and not by way of limitation. This description will clearly enable one skilled in the art to make and use the disclosure, and describes several embodiments, adaptations, variations, alternatives and uses of the disclosure, including what I presently believe is the best mode of carrying out the disclosure. Additionally, it is to be understood that the disclosure is not limited in its application to the details of construction and the arrangements of components set forth in the following description or illustrated in the drawings.

As will be apparent to those skilled in the art of designing total arthroplasty joints, the present disclosure is capable of other embodiments and of being practiced or being carried out in joints other than hip and knee joints. Specifically, while the present disclosure is described in the environment of total hip and knee replacement joints, those skilled in the art will recognize that this disclosure will be useful in any total arthroplasty joint, including knees, hips, ankles, shoulders, fingers, and the like. Also, it is to be understood that the phraseology and terminology used herein is for the purpose of description and should not be regarded as limiting.

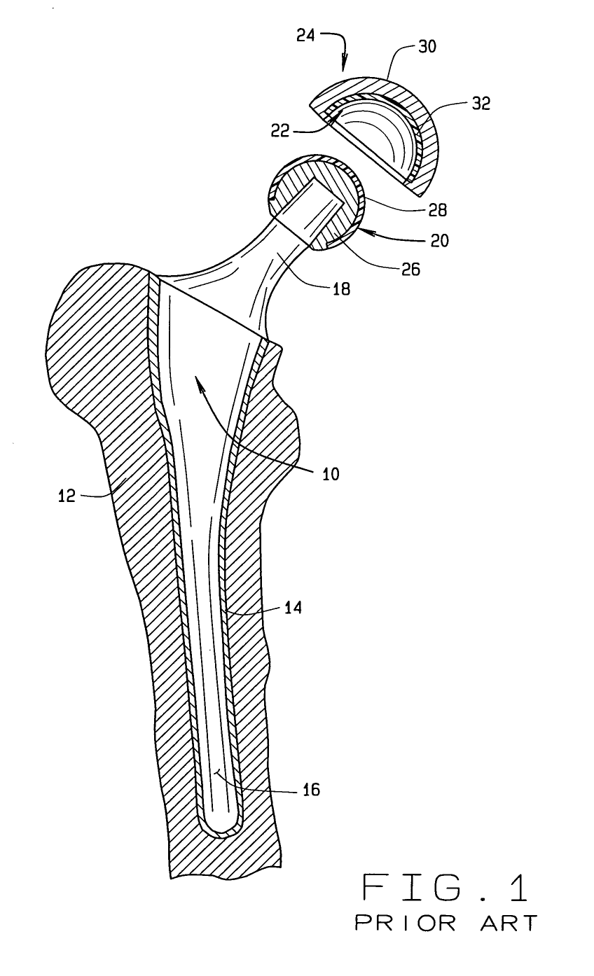

A prior art diamond coated hip joint is illustrated in FIG. 1 having a femoral component 10 that is installed in the conventional manner in a patient's femur 12. For example, the femoral component may be cemented in place by means of a suitable bone cement 14, as is well known in the art. The femoral component 10 has a femoral stem 16 inserted in femur 12. A neck 18 extends from the upper end of the stem 16. A generally spherically-shaped ball or head 20, referred to as a femoral ball, is affixed to the distal end of neck 18. While FIG. 1 is an exploded view, it will be understood that ball 20 is received (socketed) within a socket 22 of an acetabular component 24 affixed to the patient's pelvis (not shown) in the conventional manner. The femoral ball 20 has a body or substrate 26 of a durable metal, such as cobalt chromium surgical stainless steel or titanium. Ball 20 has a polycrystalline diamond coating 28 applied (e.g., sintered) to its outer surface so that the metal body of the femoral ball is the substrate for the diamond coating.

Likewise, acetabular component 24 has a similar metallic cup 30 having the part-spherical socket 22 formed therein. The surface of this part-spherical cup or socket also has a polycrystalline diamond coating 32 applied thereto so that when the femoral ball 20 is received in cup 30, the diamond coatings are in engagement with one another. The diamond coating applied to the surface of ball 20 and to the surface of cup 30 may be a polycrystalline diamond compact sintered or otherwise adhered to the surface of the substrate materials. The bearing or articulation surfaces of the joint are part-spherical in shape and rotate, move slide and roll relative to one another in a manner similar to the movement of a human hip. Full details of the hip joint shown in FIG. 1 are described in U.S. Pat. Nos. 6,610,095 and 6,800,095. As noted in the Background, such diamond coated metallic substrates for use in arthroplasty appliances experience problems with the diamond coating delaminating from the substrate due to the relative softness of the metallic substrate relative to the hard diamond coating.

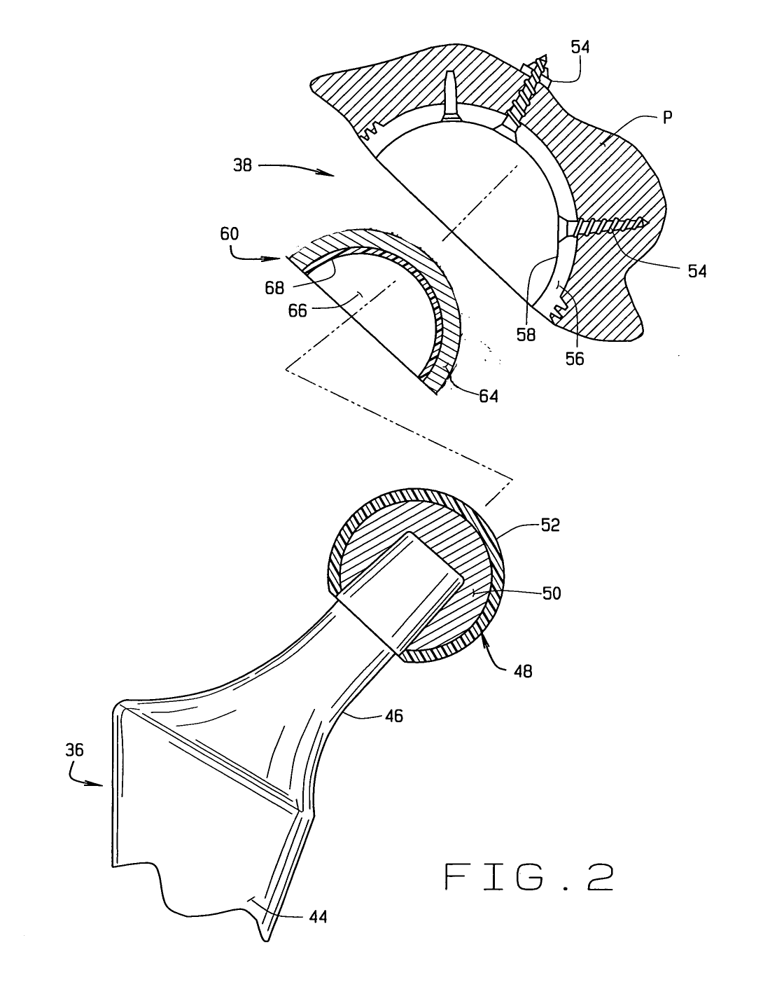

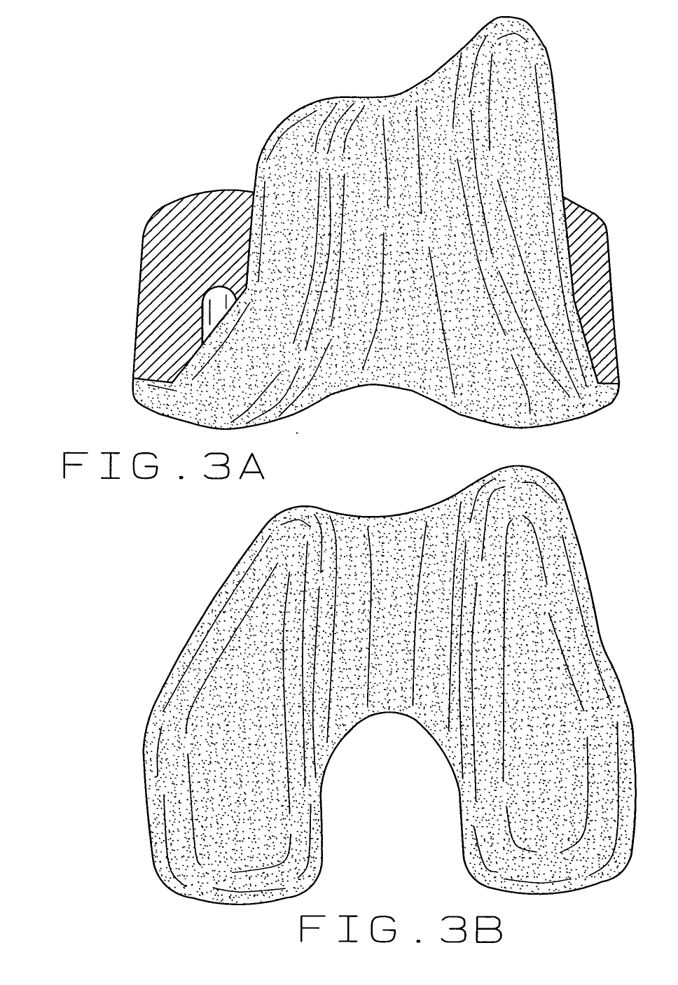

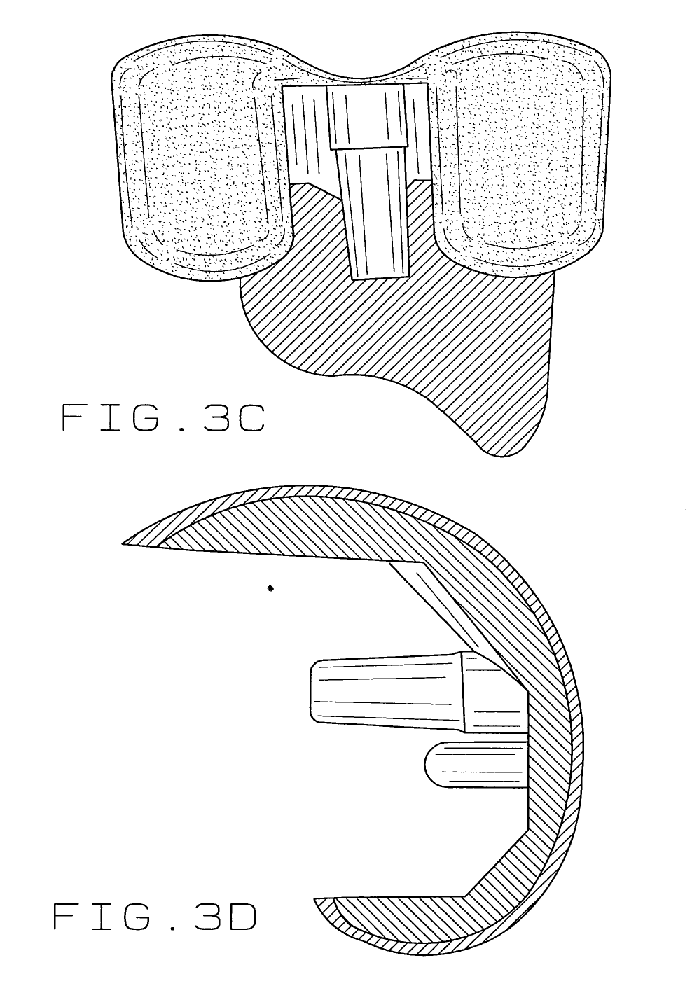

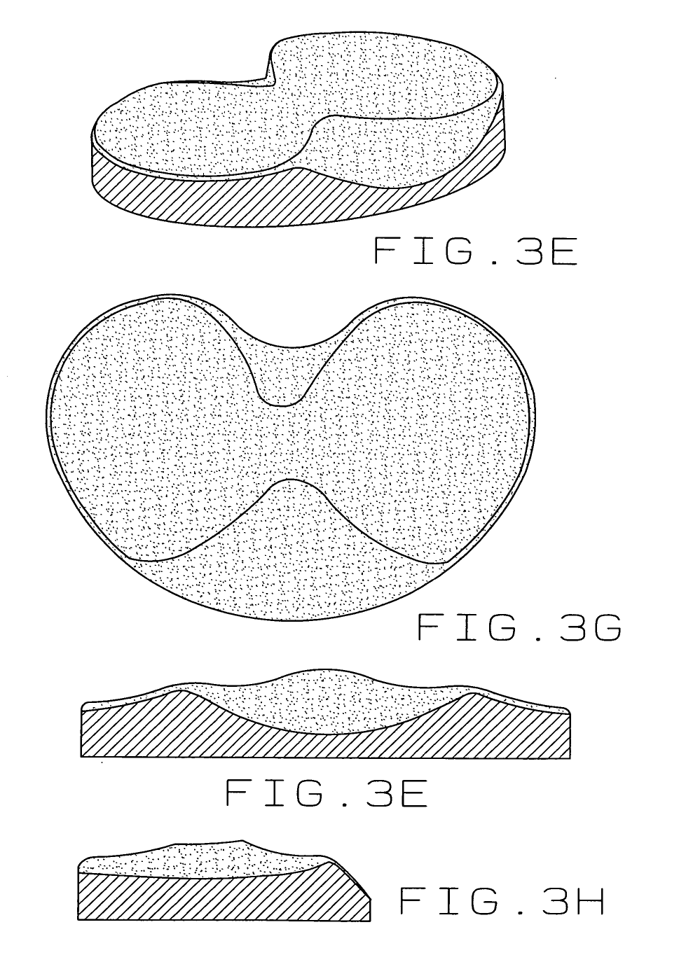

Referring to FIG. 2, one embodiment of a total joint arthroplasty of the present disclosure is shown and is indicated in its entirety at 34. Joint 34 comprises first and second bearing members 36, 38 having first and second bearing surfaces 40, 42 that are co-operable with one another or articulate against one another. While bearing members 36, 38 of joint 34 are shown in FIG. 2 to form a total hip replacement joint, those skilled in the art will recognize that this disclosure may be used with any total joint arthroplasty appliance. Therefore, the claims of this application are not limited to hip joints. As shown in FIGS. 3A-3H, an alternate embodiment of the present disclosure is shown to be a total knee arthroplasty appliance.

More specifically, the first bearing member 36 of joint 34 is illustrated in FIG. 2 and comprises a femoral component having a femoral stem 44 received in the patient's femur in the conventional manner (e.g., cemented in place). A neck 46 extends from the distal end of the femoral stem 44. The femoral component may be made of any suitable material, such as a suitable cobalt chromium surgical stainless steel or a suitable titanium alloy.

The first bearing member 36 also comprises a generally spherical femoral ball 48 is affixed to neck 46. Ball 48 has a body or substrate 50 of magnesia-stabilized zirconia ceramic material (referred to as Mg-PSZ), and has a carbon coating 52 adhered to the outer part-spherical surface (i.e., the bearing surface) of the ball 48. The femoral ball is referred to as the first bearing member 36 of joint 34 and the carbon coating 52 constitutes the first bearing surface 40. As shown in FIG. 2, the thickness of carbon coating 52 is exaggerated for purposes of clarity. It will be understood that the thickness of the carbon coating 52 is preferably very thin, ranging between about a few microns to about 100 microns, and preferably about 3-10 microns, but it could be substantially larger.

As indicated at 38, the acetabular component is surgically affixed to the patient's pelvis P by means of bone screws 54, or the like. However, those skilled in the art will recognize that there are other ways to affix the acetabular component 38 to the pelvis. Acetabular component 38 comprises an outer shell or body 56 formed of any suitable material, such as titanium. Acetabular body 56 has a part-spherical recess 58 formed therein for receiving ball 48 of the femoral component 36. A liner, as generally indicated at 60, is received in shell 56. This liner 60 may include an outer shell or cup (not shown) of a suitable metal, such as titanium. The liner 60 also includes a relatively thin liner 64 of magnesia-stabilized zirconia. Liner 64 has a female part-spherical recess 66 therein. Another carbon coating 68 is applied to the surface of the recess 66 so as to constitute a socket for receiving femoral ball 48. In one embodiment, the coating 68 covers portions of the liner 60 such as the recess 66. In another embodiment, the coating 68 may cover other portions of the liner 60 or cover the entire liner 60. As will be described, this acetabular member comprises the second bearing member 38 and the other carbon coating 68 constitutes the second bearing surface 42. It will be understood that coated liner 60 is pressed into the spherical recess 58 of shell 56.

As such, a total joint arthroplasty of an embodiment of the present disclosure comprises the first bearing member 36 and the second bearing member 38 each having articulating bearing surface 42 co-operable with one another. As least one of the bearing members 36, 38 comprise the substrate of a ceramic material, preferably magnesia-stabilized zirconia. Furthermore, at least one of the bearing members 36, 38 has a carbon coating 52, 68 applied to its respective bearing surface 40, 42. In one embodiment, the total joint arthroplasty comprises the total hip arthroplasty as shown. In this embodiment, the first bearing member 36 comprises the femoral component and the second bearing 38 comprises the acetabular component. The first bearing member 36 in the form of the femoral component has the femoral substrate 50 in the form of a head made of magnesia-stabilized zirconia and has a carbon coating applied to at least part of its bearing surface 40. The second bearing number 38 in the form of the acetabular component has a ceramic liner, preferably of magnesia stabilized zirconia, having a generally part-spherical recess therein. The recess has the other carbon coating 68 applied thereto such that when the femoral head is received within the recess, the carbon coated femoral head is socketed within the carbon coated recess.

Turning to FIGS. 3A-3H, the substrate and coatings of the present disclosure may relate to other arthroplasty appliances such as, but not limited to, knee joint appliances. FIGS. 3A-3H illustrates a total arthroplasty appliance (e.g., a knee joint appliance) the total knee arthroplasty appliance comprises the first bearing member 70 in the form of a femoral component. The femoral component 70 (FIGS. 3A-3D) has a femoral portion 72 made of magnesia-stabilized zirconia, wherein the femoral portion 72 further includes an outer surface 74. The first bearing member 70 further comprises a carbon coating 76 applied to at least part of the outer surface 74 of the femoral portion 72. In FIG. 3D, the thickness of the carbon coating 70 is exaggerated for purposes of clarity. The total knee arthroplasty appliance further comprises a second bearing member 78 (FIGS. 3E-3H) in the form of a tibial component. The second bearing member 78 has a tibial portion 80 made of magnesia-stabilized zirconia liner, wherein the tibial portion 80 includes another outer surface 82. The second bearing member 78 further comprises another carbon coating 84. In FIGS. 3E-3H, the thickness of the other carbon coating 84 is exaggerated for purposes of clarity. When the first bearing member 70 cooperates with the second bearing member 78, the carbon coating 76 contacts with the other carbon coating 80.

The total joint arthroplasty of the present disclosure comprises a first bearing member and a second bearing member each having an articulating bearing surface co-operable with one another. At least one of the bearing members comprises a substrate and has a coating applied to its bearing surface wherein the substrate has a material property comprising at least one of the following material characteristics: a hardness of about 10 GPa to about 20 GPa; a density of about 3 g/cm³ to about 7 g/cm³ and an elastic modulus of about 250 GPa to about 400 GPa. The coating has a material property comprising at least one of: a nanoindentation hardness of about 20 GPa to about 100 GPa and elastic modulus of about 170 GPa to about 1,150 GPa. Still further in an embodiment the substrate has a roughness of no more than 10 nm. Additionally, in an embodiment, the coating has a contact angle of no more than 80 degrees.

The present disclosure also relates to fabricating the total joint arthroplasty appliance having the at least two bearing members wherein each bearing member has the bearing surface adapted to be an articulating bearing relation with the corresponding bearing surface of the other joint component. The method comprises forming the bearing members of the magnesia stabilized zirconia ceramic material and applying the carbon coating to the bearing surfaces of the bearing members.

The following description describes the materials and associate parameters of the substrates and coatings of the present disclosure. Pure zirconia (i.e., zirconium dioxide, ZrO₂) normally exists in its monoclinic phase at room temperature. This crystalline structure does not form strong and tough materials, but processing with stabilizing ceramic materials, such as yttrium, the zirconium ceramic material can be produced in its tougher, tetragonal crystalline phase. Unfortunately, this material degrades in time, especially in an aqueous environment, to its monoclinic state causing grain growth which in turn leads to weakening of the material and roughening of the surface. Therefore, the commonly used yttrium-stabilized zirconia ceramics are not good candidates for use in total joint replacements that are to be carbon coated, in particular diamond coated.

Generally, zirconia ceramic material exists in a monoclinic crystalline state, which is too weak to be used for total joint certified surfaces. However, the zirconia ceramic material can be produced in a tetragonal crystalline phase, and this material is strong and tough enough to be used to produce arthroplasty surfaces. The crystalline structure must be stabilized with another metallic oxide to prevent rapid degradation to the weaker monoclinic state. Yttrium oxide is the most commonly used stabilizing material. The yttrium-stabilized zirconia ceramic material undergoes a phase transformation when it is heated, which results in a substantial change in volume that makes pure zirconia to use in practical applications. However, the addition of certain oxides, such as CaO, MgO, and Y₂O₃, into the zirconia structure results in a solid solution, which in cubic form, has little or no phase transformation during heating and cooling. This solid solution material is referred to as a stabilized zirconia.

As noted, yittra-stabilized zirconia materials tend to age and thus degrade to a monoclinic structure that occupies a larger space. This aging also results in a roughening of the surface and a weakening of the material. This has lead to increased wear and joint failure. The phase transformation also leads to weakening of the material resulting in fracture of the femoral head.

Magnesia-stabilized zirconia does not exhibit the above-noted disadvantages of yittra-stabilized zirconia. Magnesia-stabilized zirconia is heat stable, extremely strong and durable, and makes an effective material for the femoral head ball, the acetabular cup, and for other bearing members in total joint arthroplasty appliances. Such magnesia stabilized zirconia materials are commercially available from a number of suppliers, one of which is Stanford Materials Corporation of Aliso Viejo, Calif. While many different partially stabilized and fully stabilized zirconia materials are known, it is believed that different magnesia-stabilized zirconia materials will function satisfactorily.

However, one disadvantage of magnesia-stabilized zirconia is that the material does not wear well against itself, that is, if components of magnesia-stabilized zirconia are in direct bearing contact with one another, high friction and wear occurs. Heretofore, this has prevented further development in using this material for ceramic-on-ceramic bearing members in total joint arthroplasty appliances.

In accordance with this disclosure, by using two bearing members of magnesia-stabilized zirconia that have one or both of their respective bearing surfaces coated with a suitable carbon coating, the carbon coating prevents the zirconia bearing members from articulating directly on one another. The zirconia is extremely hard, as compared to prior art metallic substrate materials, and thus resists deformation upon a hard debris particle being entrapped between the bearing surfaces. Further, magnesia-stabilized zirconia has sufficient strength and toughness, and resists fracture so it makes an ideal material for joint components.

Carbon coatings such as amorphous diamond coatings are known and may be bonded to substrates in a variety of ways known in the art. Such coatings have long been used as wear coatings on rock bits in drilling for oil and gas. Further, carbon coatings such as polycrystalline diamond compacts and a variety of methods for applying polycrystalline diamond compacts to substrates are disclosed in U.S. Pat. Nos. 3,745,623; 3,767,371; 3,871,840; 3,841,852, 3,913,280; 4,311,490; 4,766,040; 5,024,680 and 6,063,149, which are incorporated herein by reference. In addition, U.S. Pat. Nos. 5,645,601; 6,610,095 and 6,800,095, which are also incorporated herein by reference, disclose the use of diamond coated metallic substrates for use in orthopedic implants. These known methods of bonding or adhering polycrystalline diamond compacts may, in accordance with the present disclosure, be used to coat the surfaces of magnesia stabilized zirconia implant components with a polycrystalline diamond coating. Such diamond coatings for use in the present disclosure are quite thin, for example having a thickness ranging between about 5 and 100 microns, and preferably about 3-10 microns thick. In the embodiments of the present disclosure, the substrate of the bearing surfaces comprises the magnesia-stabilized zirconia ceramic material. The respective coatings for the bearing members comprises a carbon coating. In one embodiment, the carbon coating is a diamond-like coating. In another embodiment the carbon coating is a diamond coating. Additionally, in one embodiment, the carbon coating is an amorphous diamond coating. Still further, in another embodiment, the carbon coating is an amorphous diamond-like coating such as but not limited to: a-C:H hard; a-C:H soft and ta-C:H. In one embodiment, the carbon coating is a polycrystalline diamond coating. Furthermore, in an embodiment, the carbon coating is a polycrystalline diamond-like coating.

Testing was conducted to evaluate the characteristics of the diamond-like coating for use as a bearing surface in joint arthroplasty. The diamond-like coating can be deposited on various substrates to improve their resistance to abrasive wear, and thus improve its wear resistance and extend its useful life as an orthopedic implant. The greater hardness of a magnesia-stabilized zirconia (Mg-PSZ; ASTM F2393) substrate will provide for a stronger coating-substrate construct than the relatively softer cobalt chromium alloy (CoCr; ASTM F799) which are also used in joint replacement. Mg-PSZ resists phase transformation and degradation in vivo and in artificial aging studies, maintaining its smooth surface finish and hardness (Trans 52^{nd} ORS, nos. 933 and 938, 2006). For comparison purposes, material were conducted on another commercially available material used in joint replacement, Oxinium (oxidized zirconium), which also has a hard and inert surface.

For the material tests, five CoCr and five Mg-PSZ never-implanted size 28 mm/+3.5 femoral heads were sent to Morgan Advanced Ceramics (Allentown, Pa.) for diamond-like coating processes. After analyzing data from a previous coated (“first-generation”) diamond-like coated Mg-PSZ head, the hardest and the thickest coating possible for these “second generation” heads were tested. The exact testing process is proprietary to Morgan Advanced Ceramics; but it is commercially available. During testing, the heads were fixed to an electrode in vacuum chamber. After a negative bias was applied to the electrode, charged gases were injected into the chamber, forming a plasma that bombards the surface to be coated with ions. The resulting diamond-like coating is generally amorphous, with a sp³/sp² ratio of between 15-25% and no long-range crystal structure. Because no heat is added during the deposition process, the substrate does not expand and dimensional tolerances are maintained. The five Mg-PSZ and the five CoCr femoral heads were coated in two separate batches, with the CoCr heads requiring an additional pre-treatment before coating began.

Several different tests have been performed to characterize the topography, mechanical properties, and overall integrity of the coatings. These tests include light microscopy and profilometry (surface finish and roughness measurement); hardness tests at the micro and nano-scales (resistance to abrasive wear); scratch tests (relative coating strength); and contact angle (a measure of whether a lubricant spreads out or beads up off the surface). The method and results for these tests are described in the following sections.

A. Light Microscopy

After cleaning the surface of each specimen by gently swabbing with 70% ethanol, the polar region of each diamond-like coating specimen was imaged under regular light microscopy at a magnification of 100×(1300×1080 pixels). Non-coated Mg-PSZ and CoCr femoral heads were also imaged for comparison. Representative scans of the femoral heads are shown in FIG. 4.

FIG. 4 illustrates light microscopy images of the typical surface of: A. uncoated CoCr; B. coated CoCr; C. uncoated Mg-PSZ; and D. coated Mg-PSZ femoral heads. Magnification=100×; bars represent 50 μm. In the CoCr images (A and B of FIG. 4), several large localized depressions can be observed along with the occasional scratch, although fewer such depressions were observed in the surface of the diamond-like coating. Mg-PSZ heads (C and D of FIG. 4) were characterized by small linear marks and the occasional larger scratch, believed to be leftover from the grinding and polishing process, which became less apparent when coated. These depressions and marks were subsequently characterized by optical profilometry. Also of note are the small dark features evident on the surface of coated specimens, which were believed to be “pinholes” in the coating, but may actually be the round positive features shown in contact atomic force microscopy (AFM) scans (see part D of this disclosure).

B. Optical Profilometry

The polar region of all femoral heads was scanned by optical profilometry using red-light phase shifting at 32×, for a scanned area of 198 μm×148 μm, and some specimens were also scanned at 10×(633 μm×476 μm). After subtracting the spherical form, the average roughness (Sa) and the root-mean-square roughness were calculated from the entire scanned area at each magnification. The Oxinium TKA femoral component was scanned at its most concave point between the condyles, with roughness parameters calculated after subtracting its second-order polynomial form.

Using an unpaired t-test, addition of the diamond-like coating did not significantly change the surface roughness of CoCr heads (p>0.09) or the Mg-PSZ heads (p>0.4). However, if one exceptionally smooth uncoated CoCr specimen was excluded, the roughness of diamond-like coated CoCr heads was significantly lower at 10× (p<0.02), suggesting that the diamond-like coating tends to fill in depressions and negative defects, resulting in a slightly smoother surface. Roughness measurements from scans at lower magnification (10×) and a larger scan area (633 μm×476 μm) were about 1.5 to 2.6 times higher than at 32×, regardless of specimen type. Table 1 illustrates roughness measurements at 32× while Table 2 illustrates roughness measurements at 10×. In one embodiment, the substrate of the present disclosure has a roughness of no more than 10 μm.

TABLE 1
Roughness measurements at 32x (198 μm × 148 μm scan area)

| Specimen | N | Sa (nm) | Sq (nm) |
|---|---|---|---|
| CoCr, no coat | 5 | 14.0 ± 6.3 | 19.6 ± 8.8 |
| Diamond-like coated | 5 | 8.02 ± 2.8 | 10.9 ± 3.4 |
| CoCr | | | |
| Mg-PSZ, no coat | 5 | 5.11 ± 0.48 | 6.39 ± 0.59 |
| Diamond-like coated | 5 | 5.30 ± 0.40 | 6.65 ± 0.46 |
| Mg-PSZ | | | |
| Oxinium TKA | 1 | 19.3 | 26.2 |

TABLE 2
Roughness measurements at 10x (633 μm × 476 μm scan area)

| Specimen | N | Sa (nm) | Sq (nm) |
|---|---|---|---|
| CoCr, no coat | 4 | 30.8 ± 8.3 | 40.8 ± 10 |
| Diamond-like coated | 2 | 21.1 ± 7.9 | 28.4 ± 13 |
| CoCr | | | |
| Mg-PSZ, no coat | 4 | 8.53 ± 1.6 | 10.9 ± 1.8 |
| Diamond-like coated | 2 | 8.12 ± 1.0 | 11.2 ± 0.92 |
| Mg-PSZ | | | |
| Oxinium TKA | 1 | 42.1 | 55.5 |

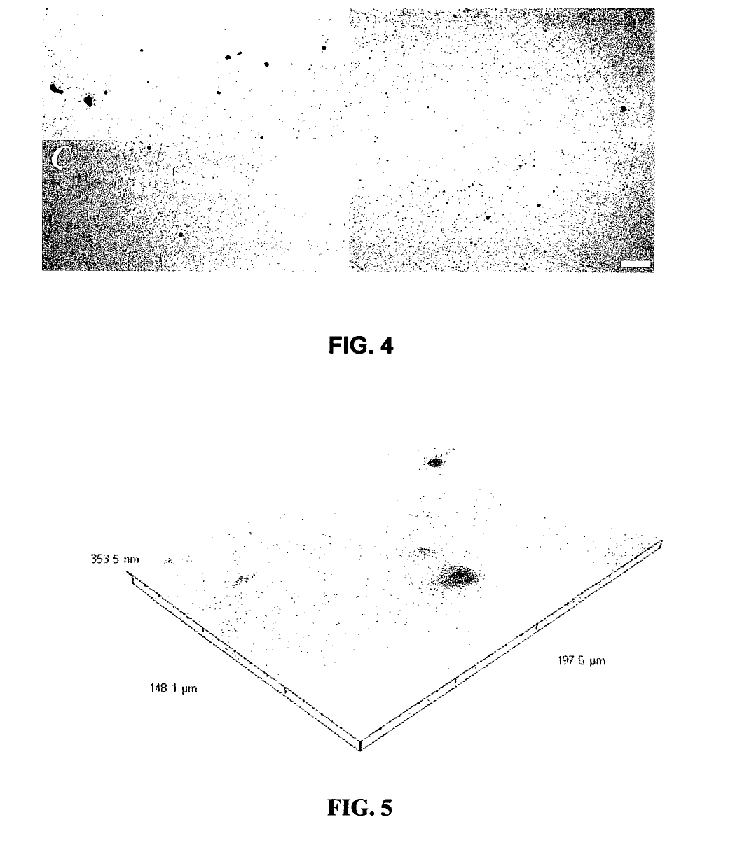

As shown FIGS. 5-9, localized depressions were more frequently observed in CoCr heads, whereas the Mg-PSZ heads had linear scratches. For both CoCr and Mg-PSZ substrates, the coating surface appears to assume the topography of its substrate, with the diamond-like coating tending to fill in depressions. While such discontinuities may represent stress concentrators on the surface, it is unlikely that the depressions or scratches penetrate the coating to the substrate, and thus should not provide an avenue for corrosive attack in

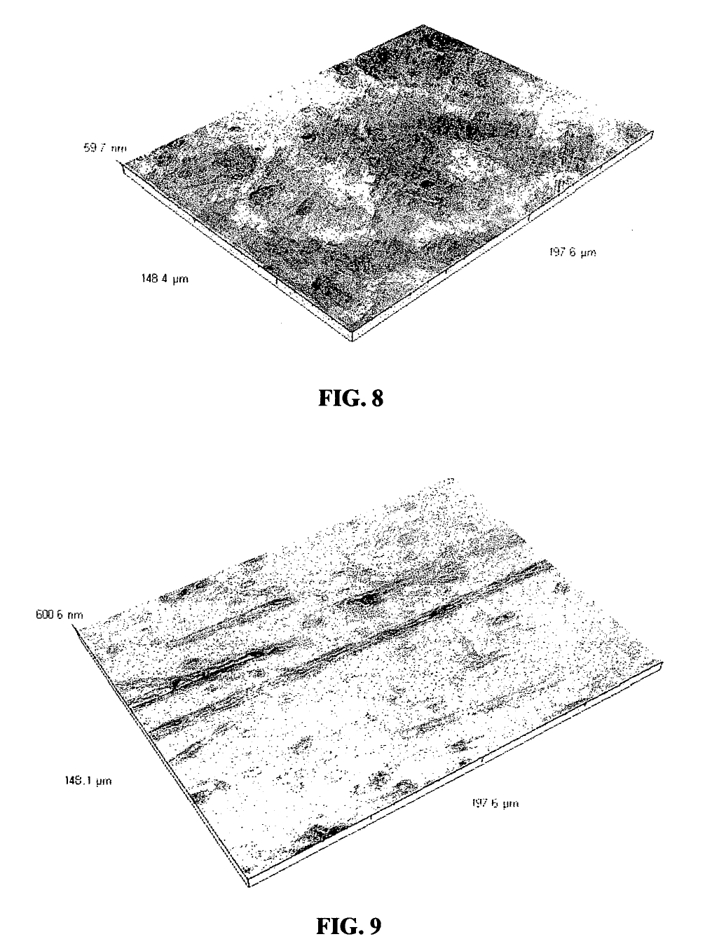

As shown in FIGS. 5-9, the following topography images (pseudo-photo surface plots) were obtained by red light phase shift measurements at 32×. For all images except the much rougher Oxinium specimen, (FIG. 9) the vertical axes were fixed at 360 nm. The vertical axis was not amplified to emphasize detail.

FIG. 5 illustrates a surface plot illustrating topography of a non-coated CoCr femoral head (Sa=18 nm), with localized depressions (diameter=20-30 μm; depth=60-100 nm) present.

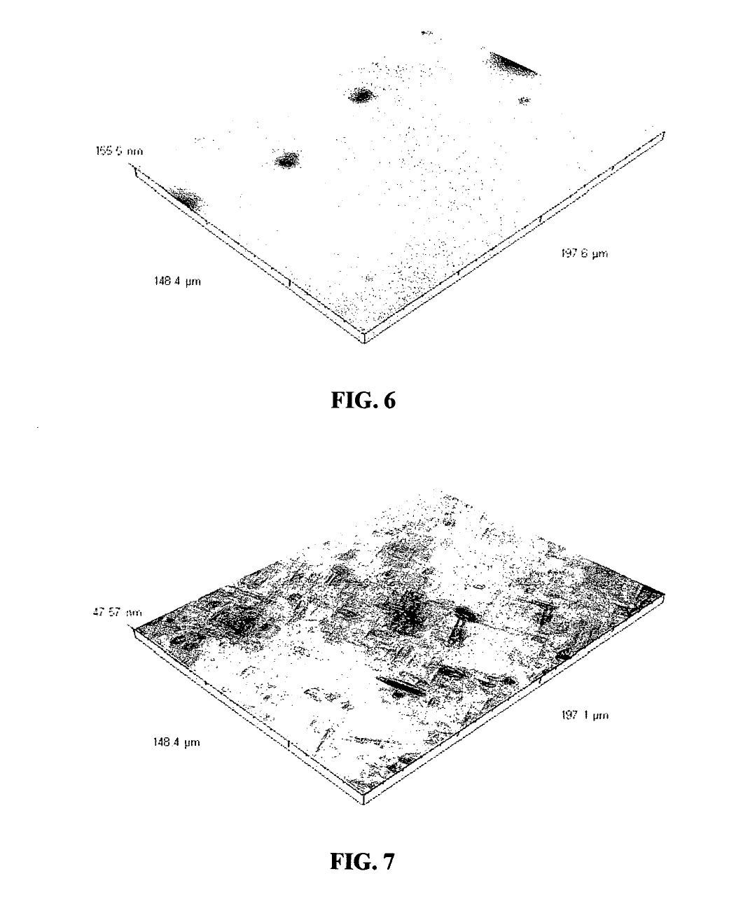

FIG. 6 illustrates a surface plot illustrating topography of a DLC-coated CoCr femoral head (Sa=12 nm), with localized depressions (diameter=10-15 μm; depth=100-125 nm) present.

FIG. 7 illustrates a surface plot illustrating topography of a non-coated Mg-PSZ femoral head (Sa=5.7 nm). The deep “scratch” has a depth of about 20 nm, is about 4 μm wide, and about 20 μm long.

FIG. 8 illustrates a surface plot illustrating topography of a diamond-like coated Mg-PSZ femoral head (Sa=5.3 nm). The most significant depression above has a conical profile but is very shallow, with a diameter of about 30 μm and a depth of about 25 nm.

FIG. 9 illustrates a surface plot illustrating topography of the articulating surface of an Oxinium TKA femoral component (Sa=19.3 nm). The surface was characterized by long scratches such as the one shown; scratches were typically about 10 μm wide and about 150 nm deep. Note that the vertical scale is 600.6 instead of 360 nm, although the vertical axis was not amplified to show detail.

C. Vickers Micro Hardness

Vickers micro hardness indentations were made at various loads in accordance with ASTM C1327, with the approach time and the dwell time each equal to 15 seconds (Table 3). Each indentation was imaged at 40× so that the diagonals could be measured for calculation of Vickers micro hardness.

Although there may be a small surface hardening effect from the polishing process, the Vickers micro hardness of uncoated femoral heads was largely independent of applied load and contact depth. As expected, the micro hardness of uncoated CoCr was significantly lower than for the uncoated Mg-PSZ ceramic. For coated heads, the composite coating/substrate hardness increases as the indentation depth decreases, as the coating hardness starts to dominate. For both CoCr and Mg-PSZ substrates, the second-generation diamond-like coating increased the hardness substantially. Table 3 illustrates the results of the Vickers tests. In one embodiment, the substrate of the present disclosure has a hardness of about 10 GPa to about 20 GPa.

TABLE 3

| | | | Theo. | | % increase |
|---|---|---|---|---|---|
| | | Applied | contact | HV | vs. |
| Specimen | n | force (N) | depth (μm) | (GPa) | uncoated |
| CoCr, no coat | 1 | 9.81 | 13.2 | 4.29 | — |
| CoCr, no coat | 1 | 4.91 | 9.31 | 4.28 | — |
| Diamond-like | 1 | 4.91 | 8.56 | 5.07 | 18% |
| coated CoCr | | | | | |
| Diamond-like | 2 | 2.94 | 6.46 | 5.35 | 25% |
| coated CoCr | | | | | |
| Mg-PSZ, no | 5 | 9.81 | 5.93 | 10.3 | — |
| coat | | | | | |
| Mg-PSZ, no | 1 | 4.91 | 8.49 | 10.6 | — |
| coat | | | | | |
| Diamond-like | 3 | 4.91 | 5.26 | 13.4 | 27% |
| coated Mg- | | | | | |
| PSZ | | | | | |
| Diamond-like | 1 | 2.94 | 3.88 | 14.8 | 40% |
| coated Mg- | | | | | |
| PSZ | | | | | |

For the diamond-like-coated heads, the applied load of coated heads had to be reduced to prevent outright coating fracture and spalling from the sharp indenter tip. However, some indentations caused fracture of the coating no matter how small the load was. These differences may be attributed to the fact that the indentations were made in a spherical object. Due to the curvature of the surface, some amount of tangential stress will always be present except for an indentation at the exact apex. In addition, pile-up (the formation of a raised lip, caused by plastic deformation) around the perimeter of the indentation may contribute to coating fracture. Pile-up is commonly found when indenting metals, and the formation of pile-up in the substrate of a diamond-like coated CoCr specimen may serve to lift the coating away from the area immediately around the indent, perhaps triggering delamination.

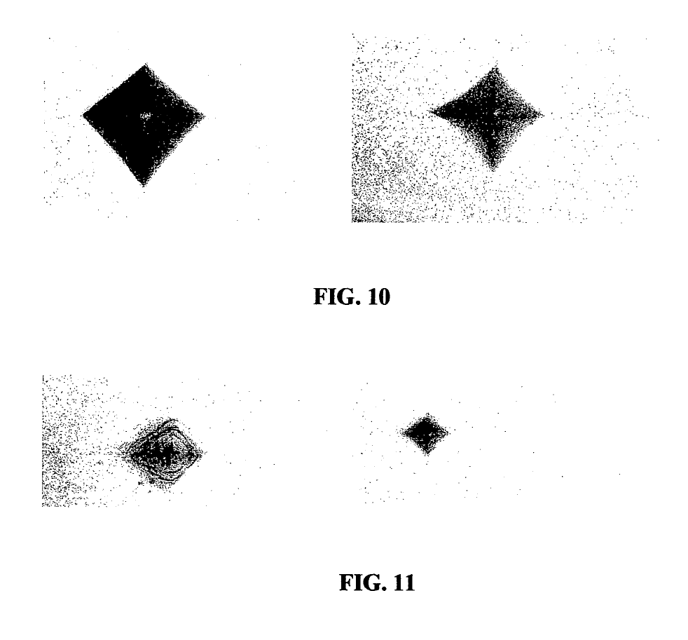

As shown in FIGS. 10-11, the hardness of the substrate affected the physical appearance of a successful indentation. FIG. 10 illustrates successful Vickers indentations into: A. uncoated CoCr and B. uncoated Mg-PSZ femoral heads, both made using a load of 4.91 N. The slight asymmetry of the indents is due to the curvature of the surface, but all indents were verified to conform to ASTM C1327. Indentations into CoCr were much more susceptible to pile-up around the perimeter of the indent. Magnification=40×; bars represent 25 μm. FIG. 11 illustrates successful Vickers indentations into: A. coated CoCr and B. coated Mg-PSZ femoral heads, both made using a load of 2.94 N. The relatively softer CoCr substrate deformed fast enough during loading to allow the coating to crack and fail cohesively, as evidenced by the successive circumferential cracks. In contrast, the coating on the Mg-PSZ head remained intact as its harder substrate deformed, with cracks rarely present. Magnification=40×; bars represent 25 μm.

D. Nanoindentation

Nanoindentation techniques have long been used to characterize the properties of thin films. The concept is similar to that of conventional micro hardness, except that the size of the indent is not measured optically. In a typical nanoindentation test, a sharp indenter tip (usually a diamond Berkovich pyramidal indenter) is loaded into the sample surface at a constant rate, held, and then unloaded at the same rate. The loading portion of the resulting force-displacement curve cannot be used, as plastic and elastic deformation occur at the same time, but stiffness can be calculated from the initial portion of the unloading curve (elastic recovery). Provided that the indenter tip geometry is properly calibrated (because even a diamond tip can start to dull), contact area and then the reduced elastic modulus (Er) and hardness (H) can be calculated.

Following a tip calibration, nanoindentation tests (TriboIndenter II, Hysitron) were performed to characterize the mechanical properties (reduced modulus Er and hardness H) of the coating independent of the substrate. Using a trapezoidal load function with loading/unloading rates of ±200 μN/s and a 20 s hold period at maximum load (2 mN), eight indents were made per specimen. At maximum load, the average indentation depth for all materials was about 300 μm deep or less; thus, the indentations likely reflected the properties of the coating only, as the coatings are believed to be greater than 3 μm thick.

The results are summarized in the Table 4 below. In one embodiment, the substrate of the present disclosure has an elastic modulus of about 250 GPa to about 400 GPa. In an embodiment, the substrate has a density of about 3 g/cm³ to about 7 g/cm³. Still further, in an embodiment, the coating of the present disclosure has a nanoindentation harshness of about 20 GPa to about 100 GPa. Additionally, in an embodiment, the coating of the present disclosure has an elastic modular of about 170 GPa to about 1150 GPa. Relative to the Vickers micro hardness data, both uncoated CoCr and uncoated Mg-PSZ exhibited a surface hardening effect (about 1.5 times harder than HV values), likely caused by the polishing process. The diamond-like coating on either substrate exhibited a lower reduced modulus than the substrate alone, but was also much harder than either substrate alone. There were no significant differences (2-sided t-test) between the properties of diamond-like coatings on CoCr and Mg-PSZ substrates. Roughness data were based on a 10 μm×10 μm contact-mode AFM scan performed on one specimen per type.

TABLE 4

| | | | hₘₐₓ | h_{final} | Eᵣ | H | Ra | Rq |
|---|---|---|---|---|---|---|---|---|
| Specimen | n | Pₘₐₓ (mN) | (nm) | (nm) | (GPa) | (GPa) | (nm) | (nm) |
| CoCr, no coat | 1 | 20.3 | 316 | 245 | 353 | 6.82 | 1.91 | 2.64 |
| Diamond-like- | 3 | 20.2 | 215 | 45.1 | 210 ± 19 | 24.5 ± 0.70 | 3.32 | 5.38 |
| CoCr | | | | | | | | |
| Mg-PSZ, no coat | 1 | 20.2 | 222 | 111 | 327 | 15.4 | 4.22 | 5.31 |
| Diamond-like | 3 | 20.2 | 225 | 42.7 | 189 ± 7.6 | 22.9 ± 0.82 | 5.35 | 7.20 |
| coated Mg-PSZ | | | | | | | | |

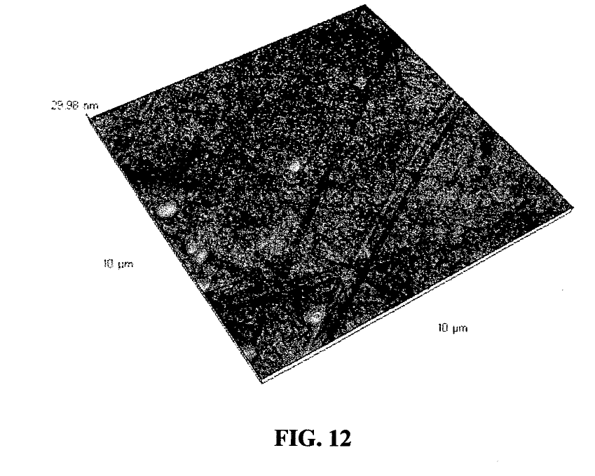

In addition to surface roughness, contact-mode AFM scans provided a detailed view of the topography of the material surfaces (FIGS. 12-15). FIGS. 12-15 illustrate pseudo-photo surface plots derived from a contact-mode AFM scan of one specimen, with a horizontal scale of 10 μm×10 μm (smaller than the images shown in part B by a factor of 292). Each AFM scan is limited to 256×256 pixels, for a lateral resolution of about 39 nm, and the vertical resolution is limited by its noise floor to about 0.2 nm. The vertical scale of each image was fixed at 100 nm, and was not amplified to show detail.

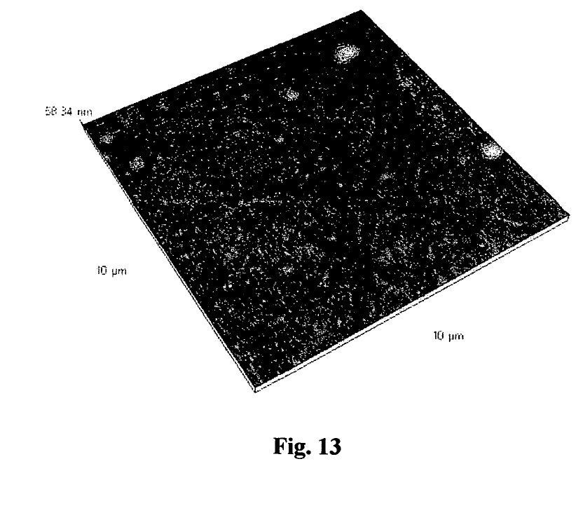

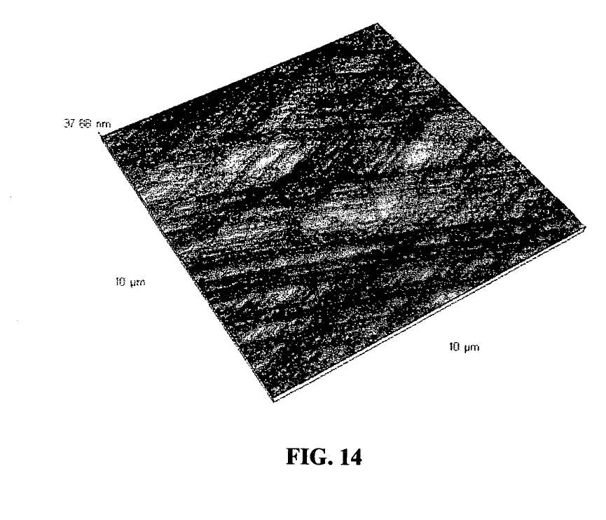

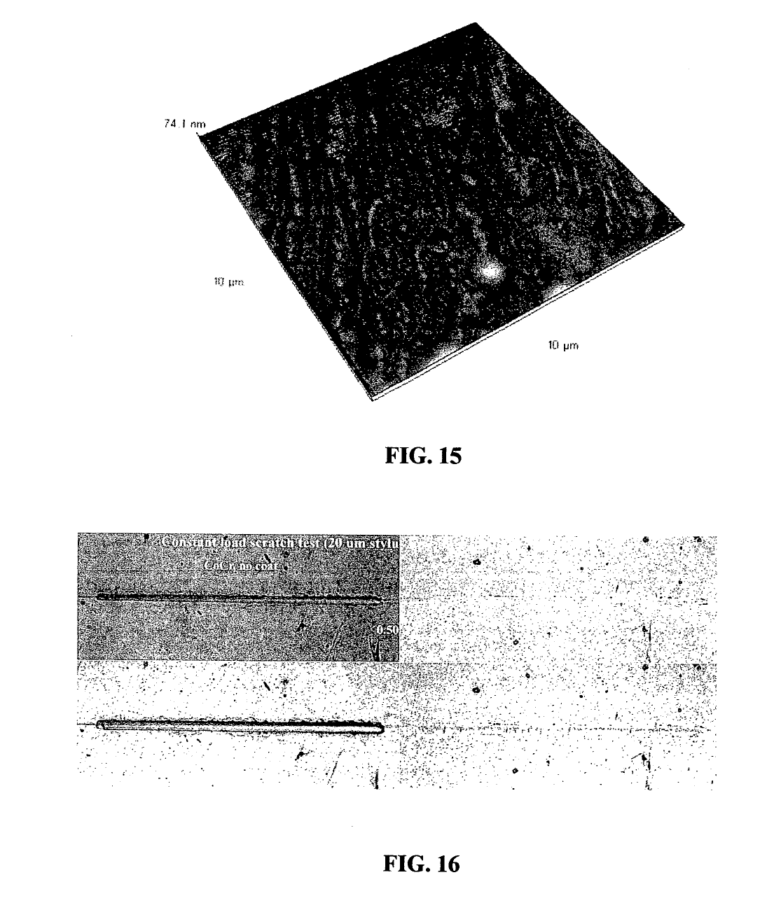

FIG. 12 illustrates a surface plot of a non-coated CoCr femoral head. FIG. 13 illustrates a surface plot of a diamond-like coated CoCr femoral head. The round positive features (each less than 50 nm high and less than 1 μm wide) were found on additional AFM scans, even after gently cleaning with alcohol, and may be caused by dust. FIG. 14 illustrates a surface plot of a non-coated Mg-PSZ femoral head. FIG. 15 illustrates a surface plot of a diamond-like coated Mg-PSZ femoral head.

E. Constant-Load Scratch Tests

1. Method and Results

Constant-load scratch tests offer a relative measure of the strength of a coating. Using a relatively sharp stylus (Rockwell C spherical diamond with tip radius=20 μm), a constant-load scratch test (scratch length=0.4 mm, scratch speed=0.4 mm/min) was performed on each sample. For each test, the starting load was 0.5 N, with the load increasing each successive pass by 0.25 N until coating fracture occurred. The scratch track was imaged by light microscopy at 200× and/or 500× between each pass. Scratch track width was calculated from the best image available as the average of three measurements (ImageJ software), viewed at twice actual size, and scratch track depth was calculated from the width and the geometry of the stylus tip.

2. Diamond-Like Coated CoCr vs. Diamond-Like Coated Mg-PSZ

Tables 5-6 summarize scratch track width and depth for each load prior to coating fracture for CoCr and Mg-PSZ substrates:

TABLE 5

| | CoCr (no coat) | | | | % |
|---|---|---|---|---|---|
| | Scratch | | Diamond-like-CoCr | | difference |
| Load | width | Theoretical | Scratch | Theoretical | in scratch |
| (N) | (μm) | depth (μm) | width (μm) | depth (μm) | width |
| 0.50 | 12.0 | 0.90 | 8.2 | 0.42 | −31.4 |
| 0.75 | 15.3 | 1.5 | 10.0 | 0.64 | −34.5 |

TABLE 6

| | Mg-PSZ (no coat) | | | | % |
|---|---|---|---|---|---|
| | Scratch | | Diamond-like-Mg-PSZ | | difference |
| Load | width | Theoretical | Scratch | Theoretical | in scratch |
| (N) | (μm) | depth (μm) | width (μm) | depth (μm) | width |
| 0.50 | 4.6 | 0.13 | 4.5 | 0.13 | −1.7 |
| 0.75 | 7.8 | 0.39 | 5.8 | 0.21 | −25.3 |
| 1.00 | 9.1 | 0.52 | 6.4 | 0.26 | −29.1 |
| 1.25 | 9.4 | 0.56 | 7.5 | 0.35 | −20.4 |

The addition of a diamond-like coating reduced the size of the scratch track by over 30% for CoCr specimens. For Mg-PSZ substrates, the width of the wear track was reduced by over 20% for all except the lowest load tested. Given the high hardness of the Mg-PSZ substrate, the scratch track width at a load of 0.5 N was very faint and difficult to measure (see following figures), especially as only 200× images were available for analysis.

The diamond-like coated Mg-PSZ heads fractured at a higher applied load (two at 1.5 N, one at 1.25 N) relative to coated CoCr heads (all fracturing at a load of 1.0 N). The “base” track width at fracture was calculated as described above, and coating thickness at coating fracture was determined from 3D non-contact profilometry scans of the scratch area (Table. 7). From a profile of the scratch, the height of the surrounding intact coating was measured relative to “plateaus” surrounding the scratch, which are believed to be areas where the coating delaminated from an undamaged substrate at coating fracture.

TABLE 7

| | | | Track width | Coating |
|---|---|---|---|---|
| | | Fracture | at fracture | thickness |
| Specimen | n | load (N) | (μm) | (μm) |
| Diamond-like- | 3 | 1.0 | 18.6 | 3.2 |
| CoCr | | | | |
| Diamond-like- | 3 | 1.4 | 11.3 | 3.5 |
| Mg-PSZ | | | | |

Comparing the diamond-like coated specimens, the physical appearances of the scratches initially and at coating fracture were strikingly different depending on the substrate. FIGS. 16-18 illustrate uncoated and coated specimens at sub-fracture loads. The CoCr substrate appeared to form a raised “lip” around the scratch, whereas the Mg-PSZ substrate eventually starts to chip but otherwise does not exhibit as much pile-up.

FIG. 16 illustrates graphical comparison of the scratch tracks of uncoated CoCr (left side) and diamond-like coated CoCr (right side), at loads of 0.50 N and 0.75 N (top to bottom). Each of the four images are about the same size (469 μm×188 μm), and were obtained by light microscopy at the same scale (200×; the bar in the corner represents 50 μm).

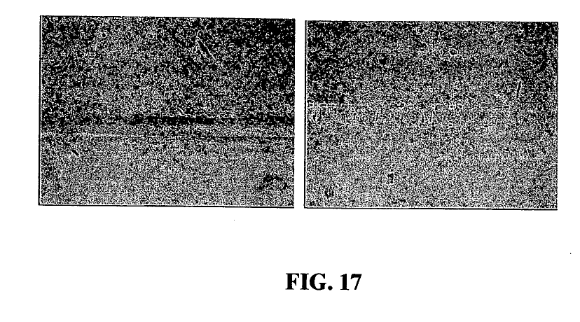

FIG. 17 illustrates a detail of scratch tracks in uncoated (left) and diamond-like coated CoCr (right) femoral heads (0.75 N load, 500×). Abundant pile-up is visible around edge of the scratch on the uncoated CoCr specimen, while small cracks (evidence of cohesive fracture) are visible in the diamond-like CoCr specimen's surface. This suggests that the substrate is yielding faster than the coating, causing the coating to be stretched apart. The diamond-like CoCr wear track is about 10 μm wide.

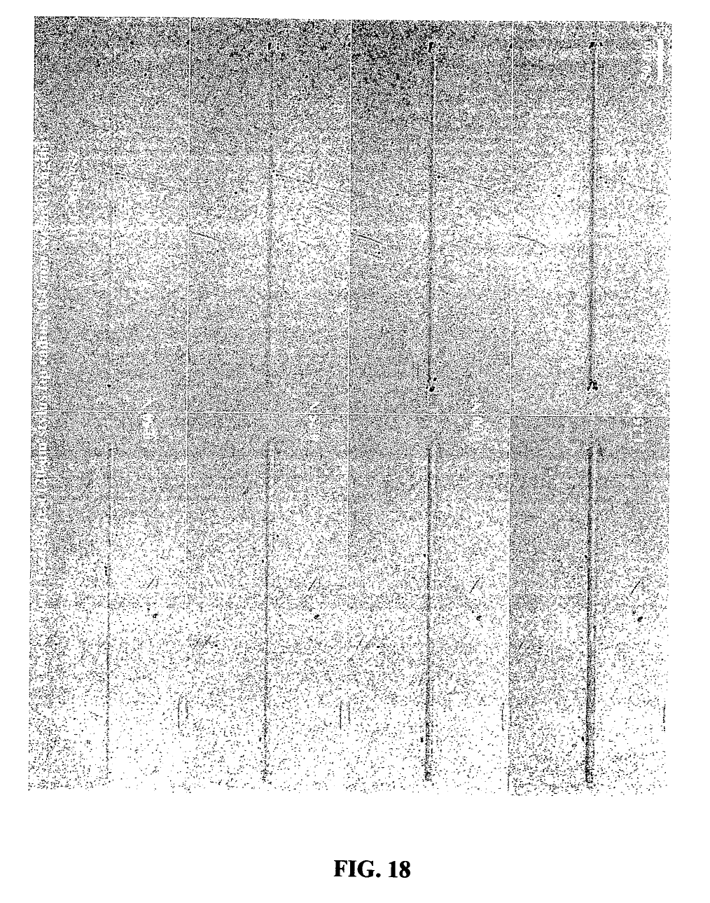

FIG. 18 illustrates a graphical comparison of the scratch tracks of uncoated Mg-PSZ (left side) and diamond-like coated Mg-PSZ (right side), at loads of 0.50 N, 0.75 N, 1.00 N, and 1.25 N (top to bottom). Each of the eight images are about the same size (469 μm×188 μm), and were obtained by light microscopy at the same scale (200×; the bar in the corner represents 50 μm).

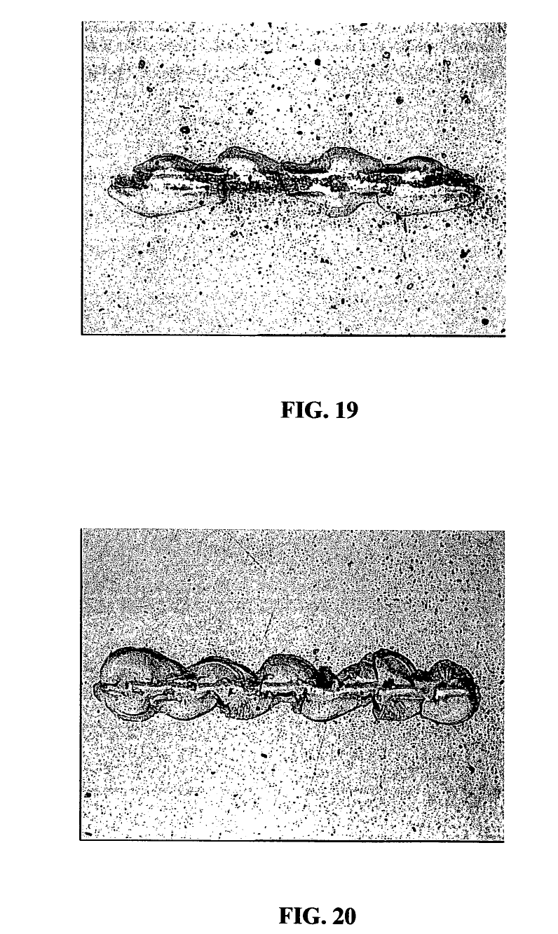

The microscopy images (FIGS. 19-20) illustrate typical wear track appearance at coating fracture. Uncoated CoCr and Mg-PSZ heads were also scratched, revealing a “lip” or pile up along the edge of the scratch track on CoCr heads but not Mg-PSZ, which on diamond-like coated CoCr specimens may serve to help initiate cracks in the coating.

FIG. 19 illustrates a diamond-like coated CoCR femoral head (light microscopy, 200×). At a load of 1 N, the coating delaminated from the substrate. The “base” of the wear track is about 19 μm wide.

FIG. 20 illustrates a diamond-like coated Mg-PSZ head at fracture

N load; light microscopy, 200×). The “base” wear track is about 11 μm wide, and appears to zig-zag across the surface, suggesting that the coating was strong enough to deflect the stylus laterally as it fractured.

3. Oxinium vs. DLC-Mg-PSZ

While diamond-like coated Mg-PSZ fractured at an average load of 1.4 N, the Oxinium specimen began to chip at a load of about 2.25 N (scratch width=38 μm), with extensive damage occurring after a load of 3 N (scratch width=50 μm). The apparently higher fracture load of the Oxinium specimen (FIG. 20) relative to the diamond-like coated specimens seems to be contradict the results of the dynamic load scratch tests (using a 200 μm radius stylus; see next section), but the Oxinium surface offered comparatively little resistance to the 20 μm radius stylus.

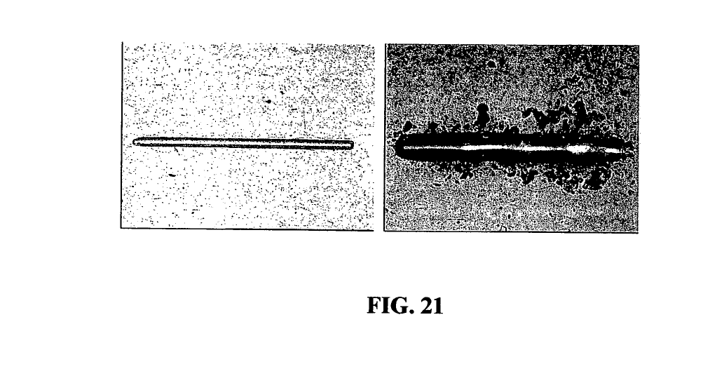

FIG. 21 illustrates a constant-load scratch tests on Oxinium TKA femoral component: left, 1 N at 200× (wear track is about 17 μm wide); right, 3.25 N at 200× (wear track is about 55 μm wide).

Unlike the diamond-like coated specimens, Oxinium is not a true coated substrate, but is rather a heavily oxidized ZrO₂ film about 5 μm thick (source: Tsai et al., Key Engng Mater 309-311:1281, 2006), caused by the diffusion of oxygen into the surface of a Zr-2.5Nb metal alloy. Thus, “delamination” of Oxinium is better described as a gradual wearing away of the surface, as opposed to delamination of a coating. The other main difference between the tests is the radius of curvature of the specimens used. The diamond-like coated femoral heads had a radius of 14 mm, while the Oxinium specimen tested were the condyies of the femoral component from a TKA, with a much larger (and more flat) radius of curvature that would not allow the small stylus tip to “dig in” under the surface as readily. It is possible that the larger 200 μm radius tip stylus used in the dynamic load scratch tests helps negate differences in specimen curvature; a larger tip is more difficult to “dig under” a coating.

Until 28 mm Oxinium femoral heads can be obtained and tested for a more “apples to apples” comparison, the best way to compare the performance of Oxinium and diamond-like coated Mg-PSZ is to compare their scratch tracks at the same applied load side by side. Table 8 compares scratch track width, measured from 200× light microscopy images, and theoretical scratch depth (based on scratch width and the 20 μm radius of the stylus) of Oxinium and diamond-like coated Mg-PSZ. For the same size load and indenter, diamond-like coated Mg-PSZ exhibited a scratch track width at least 44% smaller than Oxinium.

TABLE 8

| | Oxinium TKA | | | | % |
|---|---|---|---|---|---|
| | Scratch | | Diamond-like-Mg-PSZ | | difference |
| Load | width | Theoretical | Scratch | Theoretical | in scratch |
| (N) | (μm) | depth (μm) | width (μm) | depth (μm) | width |
| 0.50 | 8.2 | 0.42 | 4.5 | 0.13 | −44.5 |
| 0.75 | 12.4 | 0.98 | 5.8 | 0.21 | −52.8 |
| 1.00 | 17.0 | 1.9 | 6.4 | 0.26 | −62.1 |
| 1.25 | 22.5 | 3.5 | 7.5 | 0.35 | −66.8 |

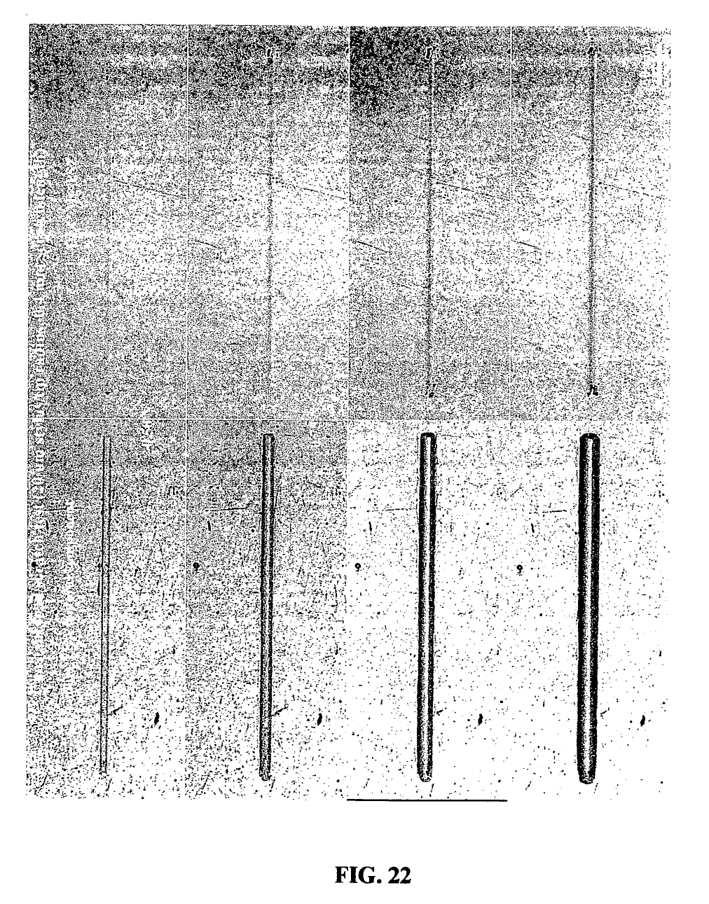

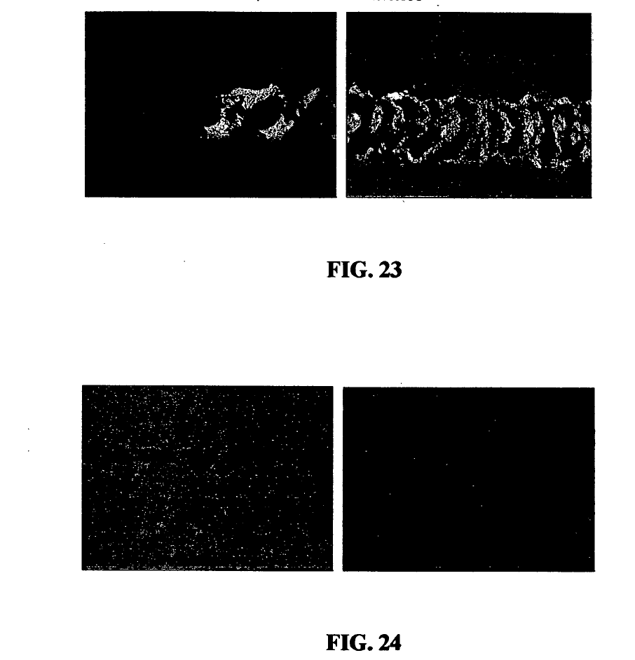

FIG. 22 illustrates a graphical comparison of the scratch tracks of Oxinium (left side) and Mg-PSZ (right side), at loads of 0.50 N, 0.75 N, 1.00 N, and 1.25 N (top to bottom). Each of the eight images are about the same size (469 μm×188 μm), and were obtained by light microscopy at the same scale (200×; the bar in the corner represents 50 μm). At the loads shown, diamond-like coated Mg-PSZ is clearly more resistant to abrasive wear than Oxinium.

F. Dynamic-Load Scratch Tests

1. Method and Results

Conventional dynamic-load scratch tests were performed as a relative measure of the strength of the coating to itself (cohesive fracture) and to the substrate (adhesive fracture), on three second-generation diamond-like coated Mg-PSZ femoral heads and three CoCr diamond-like coated femoral heads. An Oxinium femoral component used for total knee arthroplasty was also tested, to provide data from a currently produced surface-modified biomaterial for comparison purposes.

Scratch tests were performed using a Rockwell C diamond stylus with a 200 μm tip radius (ASTM C1624). For each scratch, the stylus was drawn across the surface at a constant speed while gradually increasing the applied normal force up to a maximum of L=60 N over a track length of x=6 mm, for a constant dL/dx=10 N/mm. Five scratch tests were performed on each specimen. The critical loads (L_{c}) required for cohesive and adhesive fracture were determined by light microscopy. Specimens were also scanned by optical profilometry to determine whether the scratch formed any raised edges or pile-up.

According to ASTM C1624, the definition of both cohesive and adhesive fracture depends on the relative brittleness or ductility of the coating and the substrate, and also on the thickness of the coating. Oxinium can be described as a brittle highly oxidized zirconia film (about 5 μm thick) on a ductile Zr-2.5Nb alloy substrate, and diamond-like coated CoCr is also considered to be a brittle coating on a ductile substrate (with a coating about 3.2 μm thick). For these materials, cohesive fracture was defined as the onset of chipping/wedging spallation along the scratch track, whereas adhesive fracture represented delamination of the coating from the surface. Diamond-like coated Mg-PSZ is best described as a brittle coating (about 3.5 μm thick) on a brittle substrate, with cohesive fracture represented by arc tensile cracks and adhesive fracture occurring as recovery spallation (chipping of the coating along the border of a scratch track, with the coating otherwise “pushed into” the substrate, without delamination).

TABLE 9

| | | L_{c}, cohesive fracture | L_{c}, adhesive fracture |
|---|---|---|---|
| Specimen | n | (N) | (N) |
| Diamond-like-CoCr | 3 | 9.7 ± 0.95 | 35.1 ± 0.65 |
| Diamond-like-Mg-PSZ | 3 | 41.3 ± 1.6 | 46.8 ± 2.8 |
| Oxinium (femoral | 1 | 25.5 | 34.8 |
| TKA) | | | |

As shown in Table 9, diamond-like coated Mg-PSZ ceramic required a substantially higher load to cause cohesive fracture than both Oxinium and diamond-like coated CoCr. In addition, while recovery spallation was observed, second-generation diamond-like coated Mg-PSZ did not appear to exhibit delamination at 60 N. Thus, additional scratch tests were conducted on each Mg-PSZ specimen, but with loads of 20-80 N to keep the wear track length at 6 mm and dL/dx=10 N/mm, due to the curvature of the specimen. Even at 80 N, the coating did not appear to delaminate, but instead continued to exhibit recovery spallation, with cracks and small chips along the edge of the track and appeared to be pushed into the substrate. Recovery spallation is produced by elastic recovery behind the stylus, and requires cohesive cracking of the coating and plastic deformation of the substrate. Although both the diamond-like coating and the Mg-PSZ substrate are considered brittle, the Mg-PSZ substrate has a lower hardness and a much higher elastic modulus than the diamond-like coating, allowing limited yielding of the substrate with some elastic recovery. In contrast, the CoCr substrate had a much lower hardness and a much higher elastic modulus than the diamond-like coating, for considerable plastic deformation (much faster than the diamond-like coating could deform), ultimately resulting in delamination.

FIGS. 23-26 illustrate fractures from scratch tests for the tested materials. FIG. 23 illustrates a typical cohesive fracture (left, wedging spallation at 10.6 N) and adhesive fracture (right, delamination at 38.3N) of a diamond-like coated CoCr femoral head (light microscopy, 200×). Scratch direction was from left to right.

FIG. 24 illustrates a typical cohesive fracture (left, forward tensile cracks with a small chip at 40.8 N) and adhesive fracture (right, recovery spallation at 44.6 N) of diamond-like coated Mg-PSZ femoral heads (light microscopy, 200×). Scratch direction was from left to right.

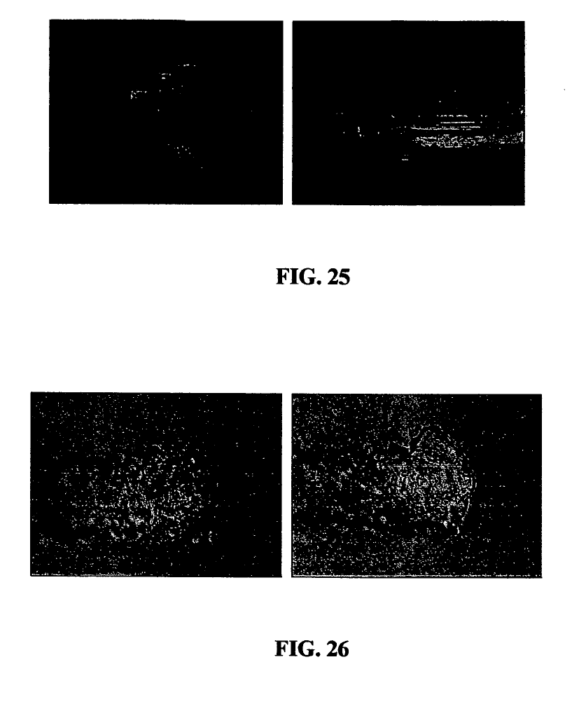

FIG. 25 illustrates a typical cohesive fracture (left, chipping at 26.8 N) and adhesive fracture (right, delamination at 38.5 N) of an Oxinium femoral component (light microscopy, 200×). Scratch direction was from left to right.

FIG. 26 illustrates a typical diamond-like coated Mg-PSZ femoral head at 80 N (light microscopy, 200×). A small amount of chipping occurred along the edges of the scratch track, but the lack of particulate debris associated with other diamond-like coating fractures (see previous constant-load scratch test figures) suggests that the coating did not delaminate, but rather was pushed into the substrate (recovery spallation). Scratch direction was from left to right.

2. Optical Profilometry of Scratch Tracks

Optical profilometry (white light interferometry) was performed to determine whether the scratch caused raised edges or pile-up along the scratch track, and also to further characterize cohesive and adhesive fracture. Due to the larger vertical range (total height) of the scratch track, red-light phase shift scans could not be performed, so all scans used white light interferometry at 32×. Scratch track scans at specific locations were obtained by finding the center of the end of the scratch (where L=60 N), and then moving the stage laterally by 3 mm to obtain scans at L=30 N. Although the specimen surfaces were curved, subsequent analyses revealed that measurement errors due to curvature was less than 1%, provided that the center of the radius of curvature was located near the highest point within the focal range of the scan.

Optical profilometry scans were analyzed with MountainsMap software to level or extract curvature and to reduce noise. Profiles were extracted from scans of different specimens at loads of 30 N and 60 N for comparison of scratch induced edge or pile-up effects, measured with respect to the overall surface. Wear scratch depth (independent of pile-up) and scratch width (widest part of scratch track; includes chipping and pile-up effects) were also measured. The results are summarized in the Tables 10 and 11.

TABLE 10

| | Approximate amount | | |
|---|---|---|---|
| | of pile-up (μm) | | |
| | | | at L = 60 N |
| | at L = 30 N | at L = 60 N | (ahead of |
| Specimen | (lateral) | (lateral) | stylus) |
| CoCr, no coat | 1.9 | 5.3 | 6.3 |
| Diamond-like- | 0.5 | 0.7 | 1.1 |
| CoCr | | | |
| Mg-PSZ, no coat | 0.4 | 0.5 | 0.6 |
| Diamond-like- | 0.2 | 0.3 | 0.5 |
| Mg-PSZ | | | |
| Oxinium TKA | 1.0 | 3.0 | 50 |

TABLE 11

| Scratch | |
|---|---|
| dimensions (μm) | |
| at L = 30 N | at L = 60 N |

| Specimen | Width | Depth | Width | Depth |
|---|---|---|---|---|
| CoCr, no coat | 120 | 5.0 | 160 | 8.0 |
| Diamond-like- | 136 | 7.6 | 240 | 12.5 |
| CoCr | | | | |
| Mg-PSZ, no | 100 | 0.7 | 105 | 2.2 |
| coat | | | | |
| Diamond-like- | 75 | 0.4 | 192 | 6.4 |
| Mg-PSZ | | | | |
| Oxinium TKA | 150 | 14.0 | 220 | 22.5 |

Among uncoated heads, Mg-PSZ exhibited pile-up an order of magnitude lower than CoCr at 30 N, and over three times lower at 60 N. Comparing the other specimens at 30 N, there were dramatic differences in scratch track depth (with Oxinium>diamond-like coated CoCr>diamond-like coated Mg-PSZ), but the differences in pile-up height were not as large. At an applied load of 60 N, profiles extracted perpendicular to the scratch direction revealed a raised edge about 3 μm high in the Oxinium specimen, while diamond-like coated CoCr exhibited substrate pile-up about 0.5 μm above the coated surface, with a “trough” of delaminated coating surrounding the scratch. If one examines just the substrate at 60 N, the scratch width and depth of diamond-like coated CoCr is almost exactly the same as uncoated CoCr. Finally, profiles extracted parallel to the scratch at L=60 N (the end of the scratch) illustrated pile-up ahead of the stylus. Again, the Oxinium specimen fared worst, with about 50 μm of pile-up, followed by diamond-like coated CoCr with substrate pile-up about 1 μm above the coated surface.

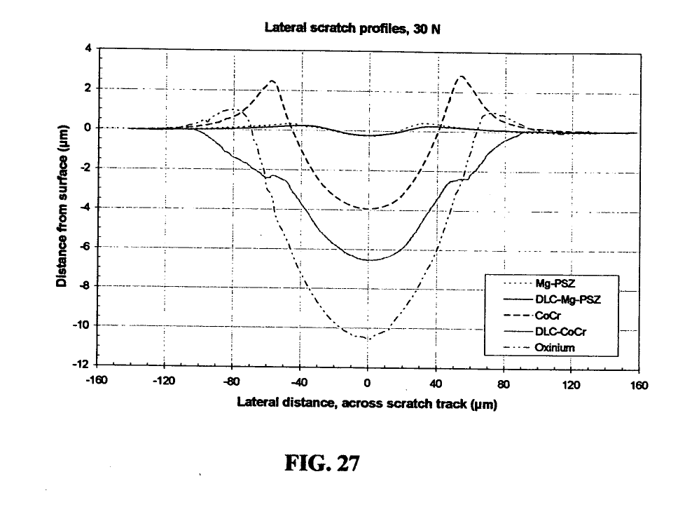

To graphically illustrate scratch width and scratch depth, along with pile-up and chipping along the scratch track, profiles were extracted from white-light interferometry scans at 16×, at locations that corresponded to loads of 30 and 60 N (the midpoint and the end of the scratch track). FIGS. 27-30 illustrate graphs for average scratch profiles at L=30 N (lateral to the scratch direction), L=30N (lateral to the scratch direction, detail), L=60 N (lateral to the scratch direction), and L=60 N (parallel to the scratch direction). Uncoated CoCr exhibited the pile-up typically seen with metals, while uncoated Mg-PSZ exhibited very little pile-up. The diamond-like coated CoCr coating had already fractured by L=30 N, exhibiting substrate pile-up that may have caused premature cracking and chipping of the coating. Diamond-like coated Mg-PSZ displayed enhanced scratch resistance relative to uncoated Mg-PSZ at 30 N, but allowed a greater scratch depth at 60 N after the diamond-like coating fractured. Finally, Oxinium also displayed significant pile-up around the edges, and its oxidized film appeared to actually peel off in front of the scratch stylus, producing about 50 μm of pile-up at the end of the scratch track.

Overall, the diamond-like coated Mg-PSZ specimen displayed the best scratch resistance relative to diamond-like coated CoCr and Oxinium, with the smallest scratch width and depth and the least amount of pile-up along the scratch edge. Because the diamond-like coated Mg-PSZ also has the highest coating strength (its coating doesn't crack until after sustaining loads that would cause delamination of diamond-like coated CoCr and Oxinium), we would expect diamond-like coated Mg-PSZ to also exhibit the best wear properties in future hip joint wear simulation tests.

FIG. 27 illustrates a scratch width and scratch depth profile, along with pile-up and chipping along the scratch track, wherein the profile was extracted from white-light interferometry scans at 16×, at a load of 30 N (lateral to the scratch direction).

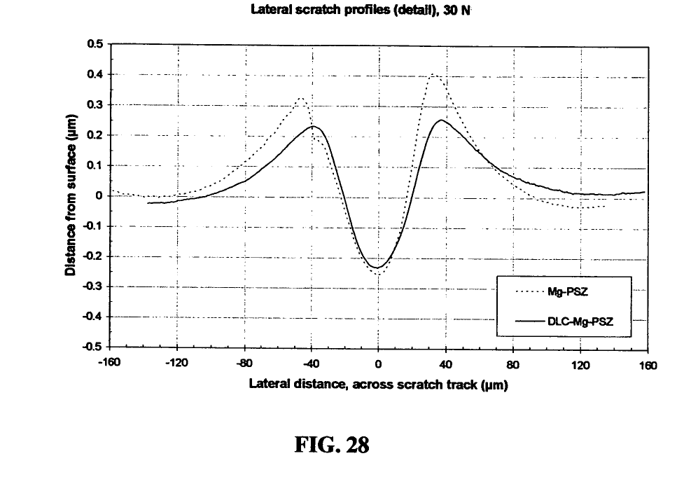

FIG. 28 illustrates a scratch width and scratch depth profile, along with pile-up and chipping along the scratch track, wherein the profile was extracted from white-light interferometry scans at 16×, at a load of 30N (lateral to the scratch direction, detail).

Average scratch track profiles extracted laterally to the scratch direction centered at an applied load of 30 N. The top figure illustrates all five materials on the same plot; below are the Mg-PSZ and diamond-like coated Mg-PSZ profiles, to illustrate detail. diamond-like coated Mg-PSZ provided the best scratch resistance (smallest scratch width and depth, with very little pile-up).

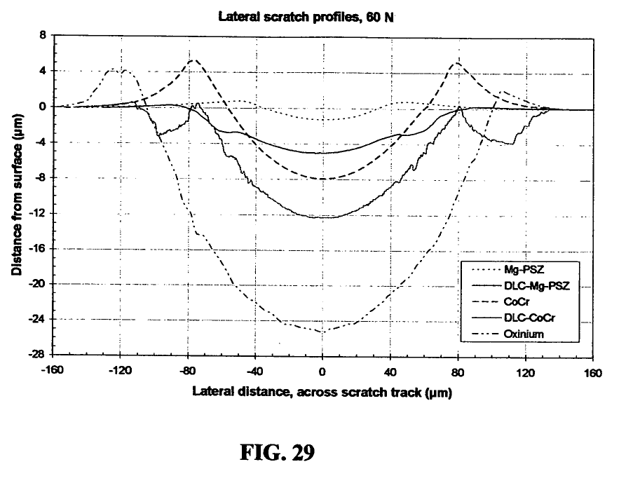

FIG. 29 illustrates a scratch width and scratch depth profile, along with pile-up and chipping along the scratch track, wherein the profile was extracted from white-light interferometry scans at 16×, at a load of 60 N (parallel to the scratch direction).

Average scratch track profiles extracted laterally to the scratch direction, centered at an applied load of 60 N. Until sustaining coating fracture at about 45 N, diamond-like coated Mg-PSZ was more scratch-resistant than uncoated Mg-PSZ. Even after coating fracture, diamond-like coated Mg-PSZ was more scratch resistant than CoCr, diamond-like coated CoCr, and Oxinium, and there was less pile-up exhibited by diamond-like coating Mg-PSZ than uncoated Mg-PSZ. Because raised edges are more likely to cause increased wear than negative features, diamond-like coated Mg-PSZ should lead to less wear than uncoated Mg-PSZ, even after coating fracture.

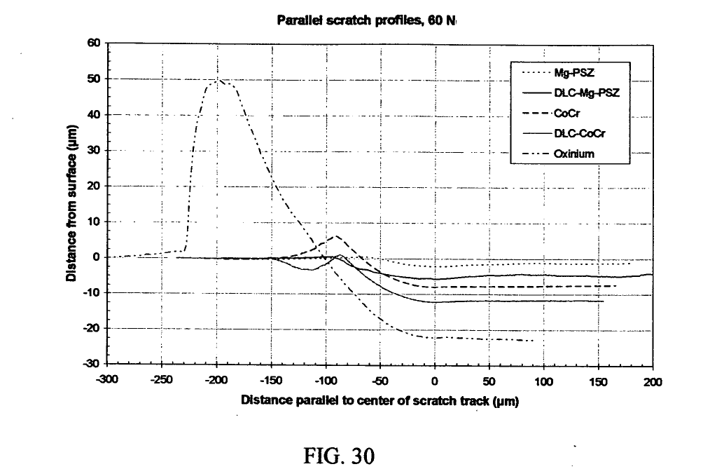

FIG. 30 illustrates a scratch width and scratch depth profile, along with pile-up and chipping along the scratch track, wherein the profile was extracted from white-light interferometry scans at 16×, at a load of 60 N (parallel to the scratch direction).

Average scratch track profiles extracted parallel to the scratch direction at an applied load of 60 N. Scratch direction was from right to left, and the y-axis of the plot is the theoretical end of the scratch track (e.g., where the center of the stylus would rest at the end of its 6 mm scratch length). Comparing uncoated specimens, Mg-PSZ exhibited little pile-up, while CoCr displayed the typical pile-up associated with metals. Among the other specimens, diamond-like coated CoCr exhibited substrate pile-up extending slightly above the coated surface, while diamond-like coated Mg-PSZ exhibited little “pile-up.” Oxinium exhibited about 50 μm of pile-up, an order of magnitude higher than even uncoated CoCr, as its oxidized film delaminated and peeled away in front of the stylus.

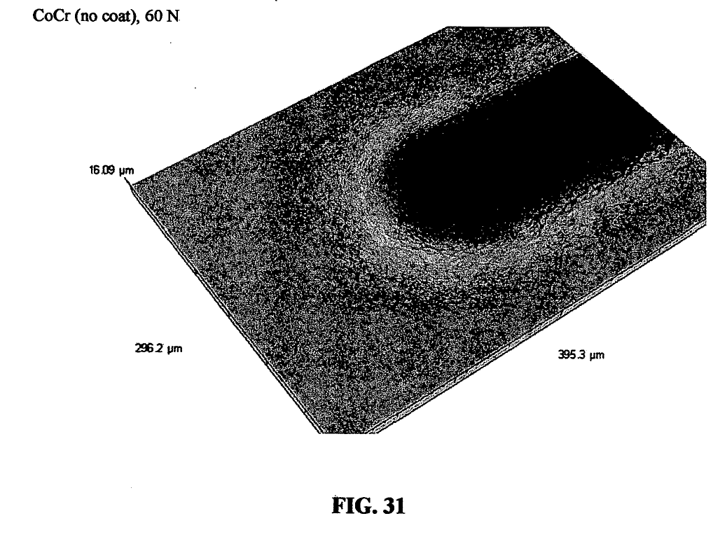

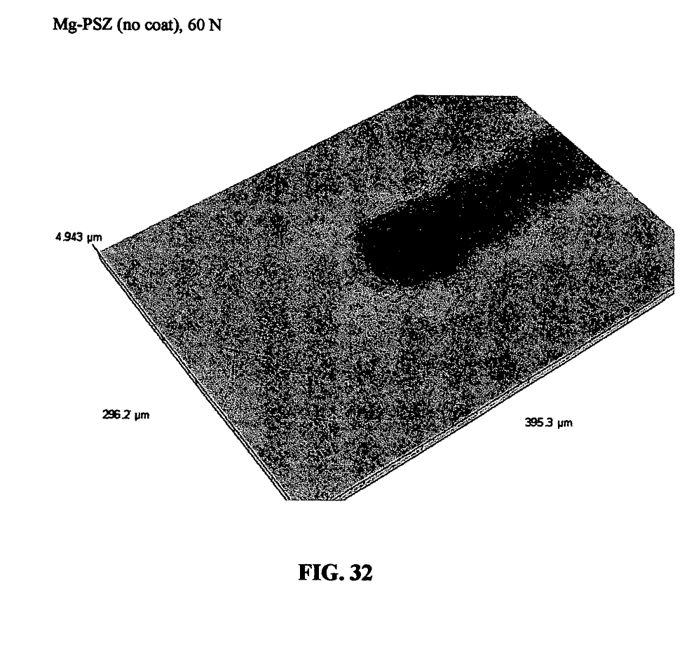

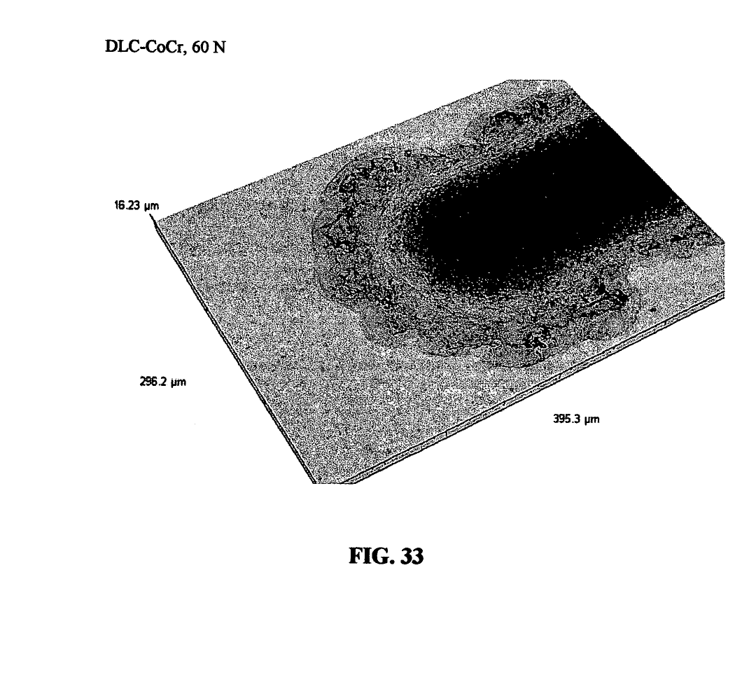

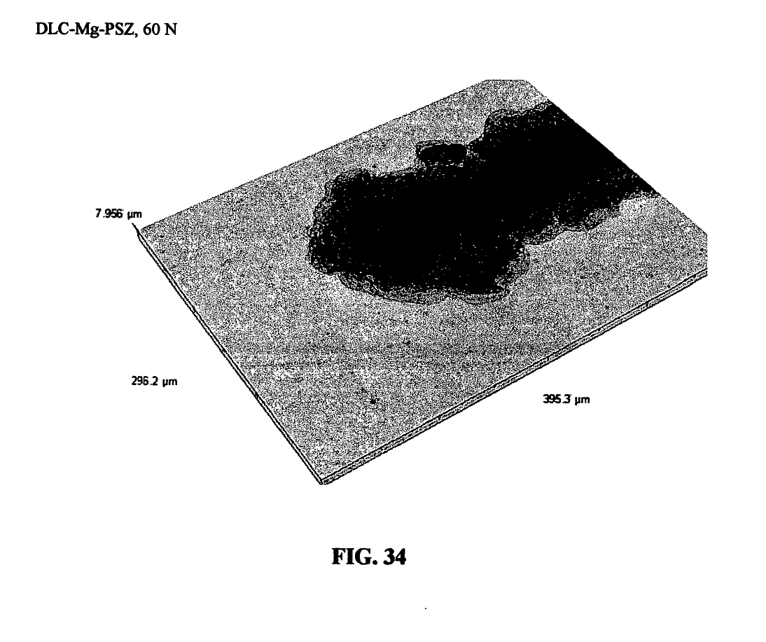

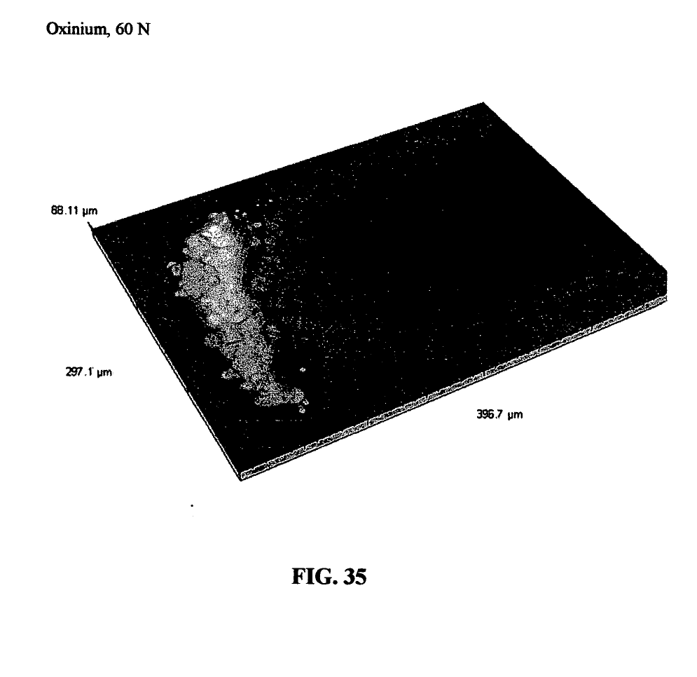

FIGS. 31-35 illustrate optical profilometry images (pseudo-photo surface plots) that were obtained by white light interferometry scans at 16×, and illustrate the pile-up at the end of the scratch (L=60 N), from which the two preceding profiles were extracted. For all images, the vertical axes were fixed at 90 μm, and were not amplified to emphasize detail. Scratch direction was from upper right to lower left.

G. Contact Angle Measurements

Contact angle is a measure of whether a surface is hydrophilic (water spreads out) or hydrophobic (water beads up). The ideal bearing surface in joint replacement should be hydrophilic, with a low contact angle, to encourage synovial fluid to cover both bearing surfaces of a joint and promote hydrodynamic lubrication, for less friction and a lower wear rate. Metals such as CoCr alloys are well-known to be hydrophobic, while ceramics tend to be more hydrophilic.

Contact angle of distilled de-ionized water was measured with a custom test setup that allowed precise placement of a droplet (0.25 μL) of water at the equator of a femoral head. Salehi et al. (Key Engng Mater 309-311:1199, 2006) had shown that bovine serum and distilled de-ionized water produced equivalent contact angles on materials used for femoral heads. Femoral heads were ultrasonically cleaned in stages, with 10 minutes in methanol, 20 minutes in acetone, and then 5 minutes in isopropyl alcohol. Each droplet was digitally imaged as a silhouette against a bright background within 5 seconds of being deposited. Glue-free cotton swabs were used to dry the specimen surface between drops. Contact angle was determined as the average of the last 10 out of 20 total droplets, to eliminate the effect of any remaining organic residue on the surface, using the NIH's ImageJ software with the LBDSA plug-in. Table 12 illustrates the results of the contact angle measurements tests. In one embodiment, the coating at the present disclosure has a contact angle of no more than 80 degrees.

TABLE 12

| | | | | Contact Angle |
|---|---|---|---|---|
| | | Contact | Difference vs. | (Salehi et al., |
| Specimen | n | Angle | uncoated (%) | 2006) |
| CoCr | 5 | 93.0 ± 1.0° | — | 75° |
| Diamond-like- | 5 | 72.3° ± 2.1° | −22.3 | 80° |
| CoCr | | | | |
| Mg-PSZ | 4 | 79.7° ± 2.4° | — | 59° (Y-TZP?) |
| Diamond-like- | 5 | 70.5° ± 1.4° | −11.6 | N/A |
| Mg-PSZ | | | | |

As shown, addition of a diamond-like coating significantly reduced the contact angle relative to the uncoated surface (p<0.00001 for CoCr, p<0.005 for Mg-PSZ). It may be possible to further reduce contact angle of diamond-like coated materials without degrading its mechanical properties by modifying the process control variables used to deposit the coating. The contact angles of CoCr, DLC-CoCr, and zirconia (Mg-PSZ) presented above are different than those reported by Salehi et al. (2006), but may reflect the influence of different surface roughness (roughness was not specified in their paper) or different material composition (e.g., their Y-TZP vs. our Mg-PSZ zirconia ceramic, or different CoCr standards). The differences could also be caused by leftover organic residue on the surface of the specimens. On every specimen, an initial increase in wettability (e.g., a lower contact angle) was observed, likely due to leftover organic residue from the cleaning process, which is why we discarded the first 10 droplets and only used the final 10 droplets to compute the average contact angle for each specimen. (Thus, the first 10 droplets of distilled water effectively helped scrub the specimen surface of any residual organic material.) Salehi et al. (2006) did not specify whether the surface was completely free of organic residue from the alcohol used as part of the cleaning process.

### III. SUMMARY

An amorphous diamond-like coating on a magnesia-stabilized zirconia ceramic substrate has been rigorously tested and outperformed other materials used for bearing surfaces in joint replacement. The use of Mg-PSZ zirconia ceramic as a substrate greatly increased the coating/substrate composite hardness and resistance to abrasive wear, relative to diamond-like coated CoCr metal substrates, and has resulted in a considerably higher hardness than possible from Mg-PSZ or CoCr alone. In standard dynamic-load scratch tests, both diamond-like coated CoCr and Oxinium delaminated before the diamond-like coated Mg-PSZ began to show cracks. In addition, Oxinium exhibited considerably higher pile-up when scratched at 60 N, up to 100 times larger than diamond-like coated Mg-PSZ at the same applied load. Because a raised edge along a scratch of an articulating surface will tend to cause additional wear and scratching of its counter face, diamond-like coating Mg-PSZ should be better suited as a bearing surface than Oxinium. Finally, the diamond-like coated Mg-PSZ substrate has a surface roughness and contact angle better than or equal to commercially available CoCr and Mg-PSZ femoral heads, which should mean less friction and wear in service. As various changes could be made in the above constructions without departing from the scope of the invention, it is intended that all matter contained in the above description or shown in the accompanying drawings shall be interpreted as illustrative and not in a limiting sense.

1. A total joint arthroplasty comprising a first bearing member and a second bearing member each having an articulating bearing surface co-operable with one another, at least one of said bearing members comprising a substrate of a magnesia-stabilized zirconia ceramic material and having a carbon coating applied to its said bearing surface.

2. The total joint arthroplasty as set forth in claim 1 wherein the carbon coating is a diamond-like coating.

3. The total joint arthroplasty as set forth in claim 1 wherein the carbon coating is a diamond coating.

4. The total joint arthroplasty as set forth in claim 1 wherein the carbon coating is an amorphous diamond-like coating.

5. The total joint arthroplasty as set forth in claim 1 wherein the carbon coating is a polycrystalline diamond-like coating.

6. The total joint arthroplasty as set forth in claim 1 wherein the substrate has a hardness of about 10 GPa to about 20 GPa.

7. The total joint arthroplasty as set forth in claim 1 wherein the substrate has a density of about 3 g/cm³ to about 7 g/cm³.

8. The total joint arthroplasty as set forth in claim 1 wherein the substrate has an elastic modulus of about 250 GPa to about 400 GPa.

9. The total joint arthroplasty as set forth in claim 1 wherein the substrate has a roughness of no more than 10 nm.

10. The total joint arthroplasty as set forth in claim 1 wherein a surface is substantially stable when installed in vivo.

11. The total joint arthroplasty as set forth in claim 1 wherein the coating has a nanoindentation hardness of about 20 GPa to about 100 GPa.

12. The total joint arthroplasty as set forth in claim 1 wherein the coating has an elastic modulus of about 170 GPa to about 1150 GPa.

13. The total joint arthroplasty as set forth in claim 1 wherein the coating has a contact angle of no more than 800.

14. A total hip arthroplasty comprising a femoral component and an acetabular component, said femoral component having a femoral head made of magnesia-stabilized zirconia and having a carbon coating applied to at least part of its outer surface, said acetabular component having a magnesia-stabilized zirconia liner having a generally part-spherical recess therein, said recess having another carbon coating applied thereto such that when said femoral head is received within said recess, said carbon coated head is socketed within said carbon coated recess.

15. The total hip arthroplasty as set forth in claim 14 wherein the carbon coating is a diamond-like coating.

16. The total hip arthroplasty as set forth in claim 14 wherein the carbon coating is a diamond coating.

17. The total hip arthroplasty as set forth in claim 14 wherein the carbon coating is an amorphous diamond-like coating.

18. The total hip arthroplasty as set forth in claim 14 wherein the carbon coating is a polycrystalline diamond-like coating.

19. The total hip arthroplasty as set forth in claim 14 wherein the other carbon coating is a diamond-like coating.

20. The total hip arthroplasty as set forth in claim 14 wherein the other carbon coating is a diamond coating.

21. The total hip arthroplasty as set forth in claim 14 wherein the other carbon coating is an amorphous diamond-like coating.

22. The total hip arthroplasty as set forth in claim 14 wherein the other carbon coating is a polycrystalline diamond coating.

23. The total hip arthroplasty as set forth in claim 14 wherein the substrate has a hardness of about 10 GPa to about 20 GPa.

24. The total hip arthroplasty as set forth in claim 14 wherein the substrate has a density of about 3 g/cm³ to about 7 g/cm³.

25. The total hip arthroplasty as set forth in claim 14 wherein the substrate has an elastic modulus of about 250 GPa to about 400 GPa.

26. The total hip arthroplasty as set forth in claim 14 wherein the substrate has a roughness of no more than 10 nm.

27. The total hip arthroplasty as set forth in claim 14 wherein a surface of the substrate is substantially stable when installed in vivo.

28. The total hip arthroplasty as set forth in claim 14 wherein the coating has a nanoindentation hardness of about 170 GPa to about 1150 GPa.

29. The total hip arthroplasty as set forth in claim 14 wherein the coating has an elastic modulus of about 175 GPa to about 250 GPa.

30. The total hip arthroplasty as set forth in claim 14 wherein the coating has a contact angle of no more than 800.

31. The total hip arthroplasty as set forth in claim 14 wherein said acetabular component comprises a metal shell adapted to be secured to the patient's pelvis, said shell having a recess therein, said liner having a metal backing, said liner being supported by said metal backing such that said metal backing and said liner may be installed as a unit within said shell recess.

32. A total knee arthroplasty comprising a femoral component, said femoral component having a portion made of magnesia-stabilized zirconia and having a carbon coating applied to at least part of its outer surface.

33. The total knee arthroplasty further comprising a tibial component, said tibial component having a magnesia-stabilized zirconia liner having a generally recessed portion therein, said recessed portion having another carbon coating applied thereto such that when said femoral portion cooperates with said recessed portion, said carbon coated femoral component engages with said carbon coated recess.

34. The total knee arthroplasty as set forth in claim 32 wherein the carbon coating is a diamond-like coating.

35. The total knee arthroplasty as set forth in claim 32 wherein the carbon coating is a diamond coating.

36. The total knee arthroplasty as set forth in claim 32 wherein the carbon coating is an amorphous diamond-like coating.

37. The total knee arthroplasty as set forth in claim 32 wherein the carbon coating is a polycrystalline diamond-like coating.

38. The total knee arthroplasty as set forth in claim 32 wherein the other carbon coating is a diamond-like coating.

39. The total knee arthroplasty as set forth in claim 32 wherein the other carbon coating is a diamond coating.

40. The total knee arthroplasty as set forth in claim 32 wherein the other carbon coating is an amorphous diamond-like coating.

41. The total knee arthroplasty as set forth in claim 32 wherein the other carbon coating is a polycrystalline diamond coating.

42. A total joint arthroplasty comprising a first bearing member and a second bearing member each having an articulating bearing surface co-operable with one another, at least one of said bearing members comprising a substrate and having a coating applied to its said bearing surface wherein the substrate has a material property comprising at least one of a hardness of about 10 GPa to about 20 GPa, a density of about 3 g/cm³ to about 7 g/cm³, and an elastic modulus of about 250 GPa to about 400 GPa and wherein the coating has a material property comprising at least one of a nanoindentation hardness of about 20 GPa to about 100 GPa and an elastic modulus of about 170 GPa to about 1,150 GPa.

43. The total joint arthroplasty as set forth in claim 42 wherein the carbon coating is a diamond-like coating.

44. The total joint arthroplasty as set forth in claim 42 wherein the carbon coating is a diamond coating.

45. The total joint arthroplasty as set forth in claim 42 wherein the carbon coating is an amorphous diamond-like coating.

46. The total joint arthroplasty as set forth in claim 42 wherein the carbon coating is a polycrystalline diamond-like coating.

47. The total joint arthroplasty as set forth in claim 42 wherein the substrate has a roughness of no more than 10 nm.

48. The total joint arthroplasty as set forth in claim 42 wherein the coating has a contact angle of no more than 80°.

49. A method of fabricating a total joint arthroplasty appliance having at least two bearing members where each bearing member has a bearing surface adapted to be in articulating bearing relation with a corresponding bearing surface of the other joint component, said method comprising the steps of:
forming said bearing members of a magnesia-stabilized zirconia ceramic material; and
applying a carbon coating to the bearing surfaces of said bearing members.
